(19) United States
(12) Patent Application Publication
(10) Pub. No.: US 2021/0115366 A1
(43) Pub. Date: Apr. 22, 2021

(54) METHODS OF MAKING IMPROVED HUMAN INTESTINAL ORGANOID COMPOSITIONS VIA APPLICATION OF STRAIN AND HUMAN INTESTINAL ORGANOID COMPOSITIONS THEREOF

(71) Applicant: Children's Hospital Medical Center, Cincinnati, OH (US)

(72) Inventors: Maxime Mickael Mahe, Cugand (FR); Michael A. Helmrath, Cincinnati, OH (US); Holly M. Poling, Cincinnati, OH (US); James Wells, Cincinnati, OH (US)

(21) Appl. No.: 16/603,611

(22) PCT Filed: Apr. 24, 2018
(86) PCT No.: PCT/US2018/029083

§ 371 Date: Oct. 8, 2019

Related U.S. Application Data
(60) Provisional application No. 62/488,984, filed on Apr. 24, 2017.

(51) Int. Cl. C12M 3/00; C12M 1/42; C12N 5/071

(52) U.S. Cl. CPC C12M 21/08; C12M 35/04; C12N 2527/00; C12N 5/0679; C12N 2513/00; C12N 5/0671

(57) ABSTRACT

Disclosed herein are methods of enhancing development of a three-dimensional in vitro model of an intestinal tissue (HIO), which may be derived from a precursor cell. The precursor cell may be, for example, an embryonic stem cell, an induced pluripotent stem cell (iPSC), or the like. The in vitro HIO model may be characterized in that the HIO has a lumen, in which a lengthening device may be inserted to promote development of the HIO. Compositions derived from the disclosed methods are also described.

This application claims priority to and benefit of 62/488,984, filed Apr. 24, 2018, entitled “Mechanically Induced Enterogenesis of Human Intestinal Organoids,” the contents of which are incorporated in its entirety for all purposes.

### STATEMENT REGARDING FEDERALLY-SPONSORED RESEARCH

This invention was made with government support under P30 DK078392 and 1K99DK110414-02 awarded by the National Institutes of Health. The government has certain rights in the invention.

### BACKGROUND

Organoids are stem cell-derived structures generated in vitro that mimic the three-dimensional architecture and physiology of an intact organ. Organoid development may allow for new approaches to modeling and studying normal development and disease processes, and introduce new approaches to medical research, drug discovery, and toxicology testing. In a typical organoid protocol, precursor cells, such as stem cells, are driven toward particular lineages through combinations of growth factors in culture media. Cells differentiate in a controlled manner to reiterate organ development starting from pluripotent stem cells in vitro. While organoid research holds considerable potential for investigating human development and disease and for advancing precision and regenerative medicine, in addition to usefulness for transplant into patients, the development of organoids is still in its infancy, and none of the currently available organoid models to date recapitulate the complete physiology of a human organ. In particular, while development of organoids has been described, advanced differentiation, increased size, maturity and function, particularly for use in transplantation, is an unmet need in the art. The instant disclosure seeks to address one or more of the aforementioned needs in the art.

### BRIEF SUMMARY

Disclosed herein are methods of enhancing development of a three-dimensional in vitro model of an intestinal tissue (HIO), which may be derived from a precursor cell. The precursor cell may be, for example, an embryonic stem cell, an induced pluripotent stem cell (iPSC), or the like. The in vitro HIO model may be characterized in that the HIO has a lumen, in which a lengthening device may be inserted to promote development of the HIO. Compositions derived from the disclosed methods are also described.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIGS. 1A-1F. Transplantation of springs into tHIOs. (1A) 28-34 day old HIOs were transplanted into the mesentery of NSG mice and allowed to grow for 8-10 weeks. Then, a second procedure was performed wherein a compressed NiTi spring was implanted inside the tHIO. Harvest occurred 14 days post spring implantation. (1B) Procedural images of the spring insertion into a tHIO. Dashed line indicates perimeter of tHIO. (1D) MicroCT of a linearly deployed spring in vivo 2 days after implantation. (1D) Schematic of springs used in experiments. Springs utilized for transplantation had a relaxed length of 12-13 mm, compressed length of 5-6 mm and an outside diameter of 2 mm. Compression of springs was maintained through use of a gelatin capsule subsequently coated with a polymer to delay deployment. (1E) Photographs of springs used in relaxed (top) and compressed/encapsulated (bottom) forms. (1F) The spring constant of those used was 1.05±0.11 N/m (n=13).

FIGS. 2A-2E. tHIO+S samples exhibit increased morphological characteristics. (2A) Representative H&E sections of tHIO, tHIO+S, infant jejunum and adult jejunum. Scale bar=50 μm. (2B) Morphometric quantification of tissue sections was plotted. Villus height, crypt depth and crypt fission were increased in tHIO+S compared to tHIO and better approximate human tissue in the cases of villus height and crypt depth. (2C) Representative pentachrome sections of tHIO, tHIO+S, infant jejunum and adult jejunum. Scale bar=100 μm. (2D) Transmission electron micrograph displaying perpendicular orientation of muscle fibers in tHIO+S, similar orientation was observed in tHIO. (2E) Quantification of muscle in pentachrome sections was plotted. Layers of circular and longitudinal muscle (CM; LM respectively) were thicker in tHIO+S compared to tHIO and trend toward that of human jejunum. n>3 for all groups, each dot represents a biological repeat and data are represented as the mean±SD.

FIGS. 3A-3G. Transcriptionally tHIO+S are matured beyond tHIO. (3A) Scaled Centered Principal Component Analysis of the tHIOs, tHIO+S, human infant and adult jejunum was performed. tHIO+S clustered closer to human jejunum tissues. (3B) Functional enrichment of the pathways upregulated in tHIO+S compared to tHIOs. (3C) Fold-change in MAPK signaling protein array. n=4 for all groups, data are represented as the mean±SD. (3D) Fold-change in ERBB receptor protein expression. n=3 for all groups, data are represented as the mean±SD. (3E) (Left panel) Fold-change in TGFβ signaling protein array. n=4 for all groups, data are represented as the mean±SD. (Right panel) Immunostaining of phosphorylated JUN in tHIO and tHIO+S. Data are representative of n=4 for all groups. (3F) Differential expression gene analysis between tHIO+S and human tissues demonstrated upregulated biological processes in the GO categories concerning intestinal development. (3G) Scaled Centered Principal Component Analysis of samples retrieved from our study and several publicly available databases.

FIGS. 4A-4E. tHIO+S samples display a shift in proliferation and expansion of the stem compartment. (4A) Double chromogenic staining for Marker of Proliferation KI67 (MKI67), red, and Cadherin 1 (CDH1), brown, on sections of tHIO, tHIO+S and adult jejunum. Scale bar=50 μm. (4B) Proliferation quantified by MKI67 and CDH1 positivity and cell position in tHIO. tHTO+S, and adult jejunum was plotted. An upward shift in proliferation is observed in tHIO+S versus tHIO. (4B′) Gaussian curve fits were plotted for each group. (4C) Normalized FPKMs were plotted for tHIO, tHIO+S and adult jejunum for cell cycle related genes MKI67, Proliferating Cell Nuclear Antigen (PCNA) and Minichromosome Maintenance Complex Component 2 (MCM2). In all cases, HIO's levels were elevated above adult jejunum. (4D) Positive expression of Olfactomedin 4 (OLFM4, gray) in immunohistochemistry sections of tHIO, tHIO+S, and adult jejunum. Scale bar=100 μm. (4E) Normalized FPKMs were plotted for tHIO, tHIO+S and adult jejunum for stem cell compartment related genes OLFM4, Leucine-rich repeat-containing G-protein coupled receptor 5 (LGR5), and BMI1 Proto-Oncogene, Polycomb Ring Finger (BMI1). OLFM4 was significantly elevated in tHIO+S compared to tHIO, while LGR5 and BMI remained similar between the engineered tissues. n>3 for all groups, each dot represents a biological repeat, proliferation data are represented mean±SD and FPKM mean±SD.

FIGS. 5A-5D. Strain's impact on secretory lineages. (5A) Sections with staining for Goblet cells (alcian blue), Paneth cells (HuLYZ), and enteroendocrine cells (CHGA) in tHIO, tHIO+S and adult jejunum. Scale bar=50 μm. (5B) Quantification of cell types in (5A). No significant differences are observed in goblet cells, though the intensity of staining visually increases toward that of adult jejunum. The number of Paneth cells is reduced in HIOs compared to adult jejunum. The number of enteroendocrine cells followed a decreasing trend and was significantly less in tHIO+S than tHIO. (5C) Normalized FPKMs were plotted for tHIO, tHIO+S and adult jejunum for the Paneth cell produced antimicrobial peptides Lysozyme (LYZ) and Alpha-Defensin 5 (DEFAS). For both, expression follows an increasing trend in tHIO+S over tHIO, while much lower than that of adult jejunum, though not significant. (5D) Normalized FPKMs were plotted for tHIO, tHIO+S and adult jejunum for enteroendocrine cell produced Serotonin (SCT) and Cholecystokinin (CCK). n>3 for all groups, each dot represents a biological repeat and data are represented mean±SD.

FIGS. 6A-6I. Epithelial integrity is retained and function improved in tHIO+S. (6A) Scanning electron micrographs of tHIO, tHIO+S, and adult jejunum epithelial surfaces. Scale bar=100 μm. (6B) Transmission electron micrographs of tHIO, tHIO+S and adult jejunum microvilli. Scale bar=1 μm. (6C) Quantification of microvilli in (6B). While both are much lower than adult jejunum, microvilli in tHIO+S are longer than tHIO. (6D) Sections with immunohistochemistry for Sucrase-Isomaltase (SI) and Dipeptidyl Peptidase 4 (DPP4) in tHIO, tHIO+S and adult jejunum. All samples displayed positivity for the brush border markers. Scale=25 μm. (6D) Normalized FPKMs were plotted for tHIO, tHIO+S and adult jejunum for SI and DPP4. Significant increases in transcripts were found in tHIO+S when compared to tHIO. (6F) Corrected short circuit current of tHIO, tHIO+S and adult jejunum was plotted. A decreasing trend is observed, but changes are not statistically significant. (6G) Corrected calculated FITC dextran flux for tHIO, tHIO+S and adult jejunum was plotted. Flux was significantly decreased in tHIO+S compared to tHIO and trended toward the level of adult jejunum. (6H) Corrected transepithelial resistance of tHIO, tHIO+S and adult jejunum was plotted. Observations across groups were similar. (6I) Normalized FPKMs were plotted for tHIO, tHIO+S and adult jejunum for tight junction components Tight Junction Protein 1 (TJP1), Junctional Adhesion Molecule 1 (F11R) and Metadherin (MTDH). For F11R and MTDH, the expression level in tHIO+S was significantly increased above that in the tHIO. n>3 for all groups, each dot represents a biological repeat and data are represented mean±SD.

FIGS. 7A-7F. Muscle function is improved in tHIO+S. (7A) Immunohistochemistry for ICCs using Anoctamin 1 (ANO1) in tHIO, tHIO+S and adult jejunum. ICCs are observed in all sample types. Arrow heads indicate ANO1+ cells. LM and CM denote longitudinal and circular muscle layers respectively. The lower right panel depicts representative recordings of spontaneous (not stimulated) muscle contractions in tHIO, tHIO+S and adult jejunum after an equilibration period. Phasic contractions related to ICC presence are measurable in all tissue types. (7B) Plotted dose response curves of tHIO, tHIO+S and adult jejunum to bethanechol. Colored arrows indicate logarithmic dose administration. (7C) The effective concentration for half the maximal response (EC50) to bethanechol was calculated for tHIO, tHIO+S and adult jejunum. For the tHIO, the EC50 could not be accurately calculated with a 95% confidence interval. For tHIO+S and adult jejunum the EC50 dosages were found to be 14.67 μM and 3.28 μM respectively. (7D) Upon removal of the adult jejunum data, the difference between the tHIO and tHIO+S EC50 curves is better observed. (7E) The maximal tension normalized per tissue mass was plotted and observed to be significantly increased in tHIO+S compared to tHIO. (7F) Plotted tension before and after administration of scopolamine in tHIO and tHO+S. Relaxation was successfully induced in both sample types. n>3 for all groups, each dot represents a biological repeat and data are represented mean±SD.

FIGS. 8A-8E. Transplantation outcomes and the sham surgery. (8A) Time of harvest pictures of sham, tHIO and tHIO+S grafts at d14. (8B) Human specific KU80 protein expression confirms tissue origins in sham, tHIO, tHIO+S and adult jejunum. tHIO in mouse kidney panel to serve as negative control; dotted line indicates tHIO graft and mouse kidney boundary below which no positive staining was observed. Scale bar=50 μm (8C) Procedural images of the sham surgery. An empty C-A-P coated capsule is inserted within the tHIO lumen and closed. Dashed line indicates perimeter of tHIO. (8D) Kaplan-Meier curve of sham and spring surgeries. Springs with a k-value of 2.7 N/m resulted in total mortality, while those with a k-value of 1 N/m had a similar survival rate as sham operated mice. (8E) Sham operated grafts displayed morphology similar to the tHIO. No significant differences in villus height, crypt depth or crypt fission were observed.

FIGS. 9A-D. Transcriptionally tHIO+S resemble human infant tissues. (9A) Unsupervised clustering based on Cosine distance and Spearman rank correlation were performed. The clustering demonstrated a higher similarity between tHIO+S and the Infant tissue. (9B) BarPlots of the top and bottom 10 genes with the highest loadings in PC1, PC2 and PC3 related to FIG. 3A. (9C-9D).

FIGS. 10A-10C. Transcriptionally tHIO+S resemble human infant tissues. (10A) Differential expression gene analysis between tHIO+S and tHIOs demonstrated upregulated biological processes in the GO categories concerning protein catabolic, biogenesis and assembly processes. Cell cycle processes were also upregulated. (10B) 254 genes were uniquely upregulated in tHIO+S compared to Human jejunum. Differential expression gene analysis between tHIO+S and human tissues demonstrated upregulated biological processes in the GO categories concerning intestinal development. (10C) 1402 genes were uniquely upregulated in tHIO when compared to human jejunum tissues. The functional enrichment highlighted biological processes involving but not limited to intracellular transports, catabolic and proteolytic processes.

FIGS. 11A-11C. Transcriptionally tHIO+S resemble human infant tissues. (11A) Scaled Centered Principal Component Analysis of the HIOs, tHIOs, tHIO+S, human infant, child and adult jejunum was performed using the 500, 2500 and 5000 genes with the highest variance across samples. tHIO+S clustered closer to human fetal and infant jejunum tissues. (11B-11D) BarPlots of the top and bottom 10 genes with the highest loadings in PC1, PC2 and PC3 related to FIG. 3G. (11E-11F) Functional enrichment analysis of the principal component loadings accounting for the biological processes seen in the PCA (FIG. 3G).

FIGS. 12A-12E. MAPK, EGFR and TGFβ signaling in tHIO and tHIO+S. (12A) MAPK signaling membrane map and developed chemiluminescent membranes for tHIO and tHIO+S. (12B) Quantification of proteins in the MAPK array. (12C) EGFR signaling membrane map and developed chemiluminescent membranes for tHIO and tHIO+S. (12D) Quantification of proteins in the ERBB array. (12E) TGFβ signaling membrane map and developed chemiluminescent membranes for tHIO and tHIO+S. (12F) Quantification of proteins in the TGFβ array.

FIGS. 13A-13C. Sham epithelial proliferation is similar to tHIO. (13A) Total proliferation within the crypt as a percentage is similar between experimental groups. (13B) Double chromogenic staining for Marker of Proliferation KI67 (MKI67), red, and Cadherin 1 (CDH1), brown, on section sham tissue. Scale bar=50 μm. (13C) Proliferation quantified by MKI67 and CDH1 positivity and cell position in sham tissue was plotted. (13C′) Gaussian curve fits for proliferation by position in sham and tHIO groups were plotted and similar.

FIGS. 14A-14E. Using chamber correction factor and uncorrected data. (14A) Density of microvilli in tHIO, tHIO+S and adult jejunum was plotted. (14B) Correction factors for Using chamber experiments were calculated based upon the combined surface area of a tube and hemisphere which approximate the surface area of a villus. Calculated correction factors based upon morphometric measurements for the tHIO, tHIO+S and adult jejunum groups. (14C) Short circuit current of tHIO, tHIO+S and adult jejunum was plotted. (14D) Calculated FITC dextran flux for tHIO, tHIO+S and adult jejunum was plotted. (14E) Transepithelial resistance of tHIO. tHIO+S and adult jejunum was plotted.

FIGS. 15A-15D. Muscle contraction in tHIO+S compared to human tissues. (a5A) Representative recordings of spontaneous (not stimulated) muscle contractions in tHIO, tHIO+S, infant and adult jejunum after an equilibration period. (15B) Muscarinic receptors mRNA expression in tHIO, tHIO+S, infant and adult jejunum. (15C) Maximal tension after bethanecol stimulation normalized per tissue mass in tHIO, tHIO+S compared to infant and adult jejunum. (15D) Plotted tension before and after administration of scopolamine in tHIO, tHIO+S, infant and adult jejunum. Relaxation was successfully induced in all sample types. n>2 for all groups, each dot represents a biological repeat and data are represented mean±SD.

FIGS. 16A-16B. Microdissection set up and resulting tissues. (16A) The set up for microdissection of epithelium is pictured, with critical supplies labeled. (16B) Toluidine Blue stained thick sections of tHIOs after demounting from hemi-chambers of the Using apparatus upon completion of the permeability assay. Scale bar=100 μm.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, for example within 5-fold, or within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

As used herein, the term “pluripotent stem cells (PSCs)” encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes. hiPSC refers to human iPSCs. In some embodiments, iPSCs may be derived by transfection of certain stem cell-associated genes into non-pluripotent cells, such as adult fibroblasts. Transfection may be achieved through viral vectors, such as retroviruses. Transfected genes may include the master transcriptional regulators Oct-3/4 (Pouf51) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some embodiments, a retroviral system is used to transform human fibroblasts intopluripotent stem cells using four pivotal genes: Oct3/4, Sox2, Klf4, and c-Myc. In alternative embodiments, a lentiviral system is used to transform somatic cells with OCT4, SOX2, NANOG, and LIN28. Genes whose expression are induced in iPSCs include but are not limited to Oct-3/4 (e.g., Pou5fl); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, and LIN28.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In both directing and fostering the natural ability of stem cells to self-organize, significant advances have been made in the generation of functional human intestinal organoids. However, conventional methods for their generation are solely biological, when indeed intestinal development and morphogenesis are impacted by dynamic mechanical forces. As understanding of these mechanical forces during development deepens, the opportunity to include them in tissue engineering strategies arises. Here, Applicant has generated intestinal tissue and incorporated uniaxial strain serving to induce growth and maturation of the tissue. Using a variety of outcome measures including morphometric quantification, transcriptome profiling, and functional assays, Applicant found the newly generated tissue to be more similar to native human intestine after strain exposure. The size and complexity of the tissue was significantly improved, as was muscle tone. The novel methods incorporate a developmentally relevant mechanical cue in the development of human intestinal tissue and results in enhanced maturation and enterogenesis.

The establishment of a three-dimensional in vitro model of the human intestine has required a deep understanding of endoderm and intestinal development.¹ These complex structures are created from human embryonic stem cells and/or induced pluripotent stem cells (PSCs) by the perturbation of signaling pathways through a temporal series of growth factor manipulations^{2, 3}. This exclusively biological and mechanically static methodology to intestinal tissue generation has been successful in creating both functional intestinal lineages (e.g., Paneth, Goblet, enteroendocrine, and enterocyte) and architecture similar to that of native intestine (e.g. crypts, villi, and smooth muscle layers)^{2, 4}. These tissues, termed human intestinal organoids (HIOs) have been shown to be functional and possess the ability to engraft in vivo⁴. However, the maturation status of transplanted HIOs (tHIOs) best approximates that of human fetal intestinal tissue, which ultimately limits their utility⁶. Current methods have yielded a powerful tool for use in both basic science and clinical applications. HIOs are an avenue to study intricate physiological interactions and personalize medicine for patients across the globe^{7,8}. However, in order to build upon these uses it is imperative that tissues with increased size, maturity and function are generated, because creating tissue for transplantation remains an unmet clinical need.

Recently, there has been a renewed interest in not only the biological cues impacting development and morphogenesis, but the mechanics of development as well⁹. In particular, Shyer and Savin have elegantly demonstrated that strain plays a role in intestinal development. Gut looping, villification, and the localization of intestinal stem cells to the crypt have all been associated with mechanical strain¹⁰⁻¹³. As strain is a common contributing factor between several architectural features of the intestine, it was discovered by Applicant that incorporating this pulling or tension force within the generation of HIOs could advance differentiation, and that both mechanical and biological cues could prove advantageous in the generation of larger scale tissues with a maturation status beyond that of human fetal intestine.

In order to incorporate strain into the current protocol of HIO generation, Applicant combined in vivo transplantation with the repurposing of a lengthening device designed for the treatment of short bowel syndrome: the spring. Much like other endoluminal lengthening devices, the spring has been shown to stimulate an adaptive morphometric response in the setting of mature tissues, though additional effects have not been thoroughly characterized¹⁴⁻¹⁸. In one aspect, the use of a spring may be advantageous because its geometry and applied force may be scaled and may allow for the accumulation of mucous as the tHIO is a closed system¹⁴⁻¹⁷. In combining these transplantation strategies, Applicant found that the application of strain in a fetal setting is capable of eliciting tissue maturation and enhancing overall growth of the tHIO. Further, Applicant found that, by including a lengthening device, for example, a spring, into the lumen of an organoid, improved development of the organoid can be achieved. In particular, Applicant found that linear deployment of a spring having a suitable spring constant k was an important consideration in achieving this effect. In particular, Applicant has found that a spring constant that is too high will deploy in a nonlinear fashion and will cause obstructions resulting in the death of the host. If the spring constant k is too low, the spring/lengthening device will be too soft and the tissue will not experience sufficient strain.

Applicant has combined a common mechanic of development, uniaxial strain, with the generation of HIOs. Grafts that had undergone strain (tHIO+S) were found to have increased intestinal and maturation features compared to those that did not experience applied strain, including transcriptional, morphological, and functional shifts toward postnatal human intestine. Disclosed herein is the first description of mechanically manipulating tHIOs in vivo to result in the successful induction of maturation and enterogenesis.

In one aspect, a method of enhancing development of a three-dimensional in vitro model of an intestinal tissue (HIO) derived from a precursor cell is disclosed. The precursor cell may be, for example, an embryonic stem cell (H1 line), an induced pluripotent stem cell (iPSC), or the like. The in vitro HIO model may be characterized in that the HIO has a lumen, which method further comprises the step of inserting a lengthening device into the lumen, which applies strain to the lumen.

In one aspect, the lengthening device may comprise a spring, for example, in one aspect, a nickel titanium (nitinol, NiTi) spring. In one aspect, the lengthening device may have a relaxed length of about 10 to about 15 mm, and may have a compressed length of about 4 to about 8 mm. In certain aspects, the lengthening device may have a diameter of about 1 to about 3 mm, or about 2 mm. The lengthening device, in some aspects, may a spring constant of from about 0.5 N/m to about 2 N/m, or from about 0.7 to about 1.7, or from about 0.9 to about 1.2 or about 1 N/m. The lengthening device may be contained in a degradable coating, for example, for a degradable coating that maintains the compressed length until deployment. An exemplary coating includes a gelatin capsule, which may further comprise a polymer in an amount and of a type sufficient to delay deployment. In one aspect, the lengthening device may comprise an enteric coating, for example, an enteric coating of cellulose acetate phthalate (C-A-P). The lengthening device may be one which provides a cavity sufficient to allow flow of lumen secretions. Other suitable devices will be readily appreciated by one of skill in the art.

In one aspect, the method may comprise a first and a second engraftment period. The first engraftment period may comprise the step of transplanting the HIO into an immune compromised animal model prior to implantation of the lengthening device, for example, in the mesentery of the animal model. The first engraftment period may be carried out for a period of time sufficient for said develop crypt regions, villi, and smooth muscle layers. In one aspect, the method may comprise the step of a second engraftment period occurs, which occurs after the first engraftment period. The second engraftment period may comprise the steps of inserting the lengthening device after the first engraftment period for a period of time sufficient to allow increased villus height and crypt depth as compared to a control HIO not subjected to said lengthening device. In one aspect, the first and second engraftment period may be for a period of time sufficient to allow the HIO to have increased villus height and crypt depth and crypt fission, and increased longitudinal and circular muscle thickness, as compared to a control HIO which does not contain a lengthening device.

In one aspect, the first engraftment period may be of a period of time of from about 6 to about 14 weeks, or from about 10 to about 12 weeks, or for a period of time sufficient to allow said HIO to obtain a blood supply and/or grow to a size sufficient for implantation of the lengthening device, for example, from about 5 mm to about 2 cm in length, or from about 10 mm to about 1.5 cm in length.

In one aspect, the precursor cell is a pluripotent stem cell or an induced pluripotent stem cell, for example, an embryonic stem cell. In one aspect, the HIO may be derived from a pluripotent stem cell, wherein said pluripotent stem cell is derived from a fetal tissue stem cell.

In one aspect, three-dimensional human intestinal organoid (HIO) composition derived from a pre-cursor cell in vitro are disclosed. The HIOs may comprise a lengthening device as described above.

In one aspect, three-dimensional human intestinal organoid (HIO) composition derived from a pre-cursor cell in vitro is disclosed, wherein said HIO is lacking one or more features native to a fully developed organ, for example, wherein said HIO is devoid of a vascular system.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

Nitinol Spring as an Endoluminal Lengthening Device for tHIOs.

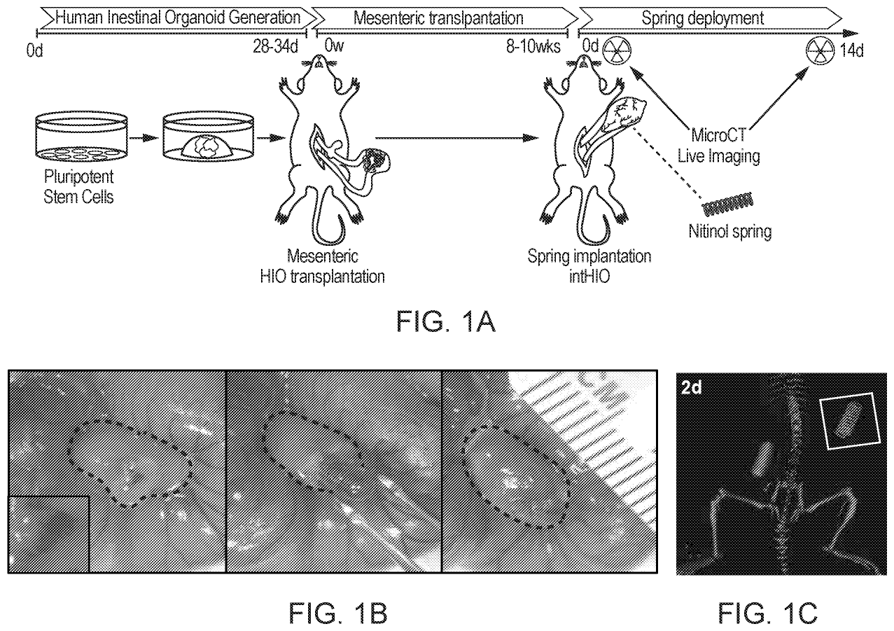

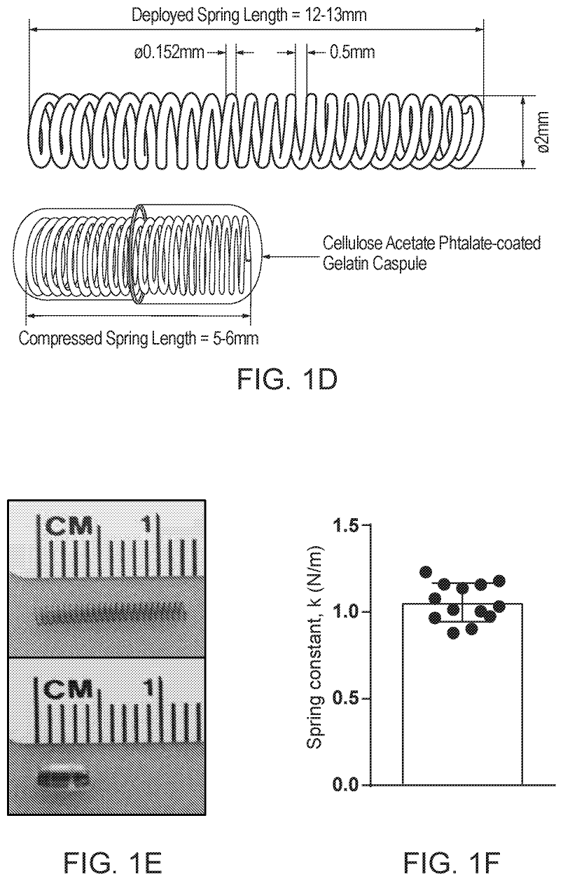

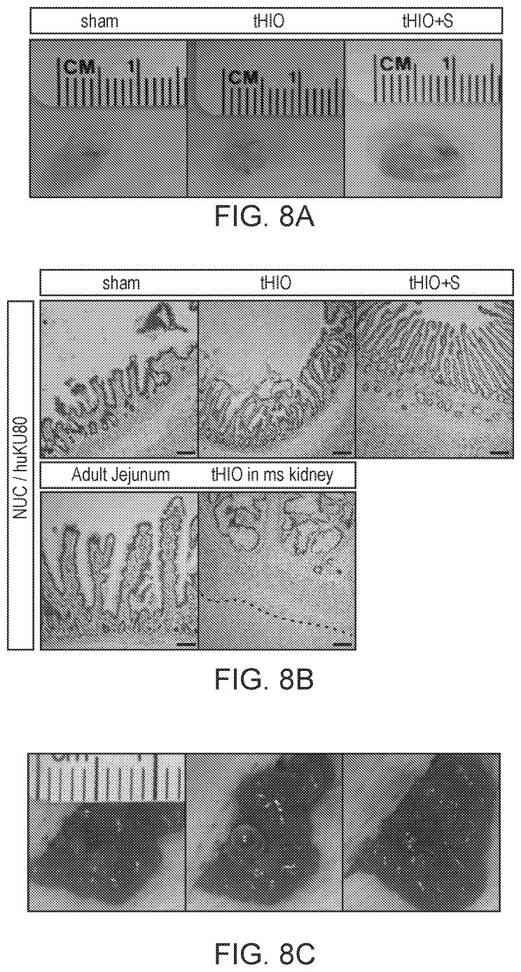

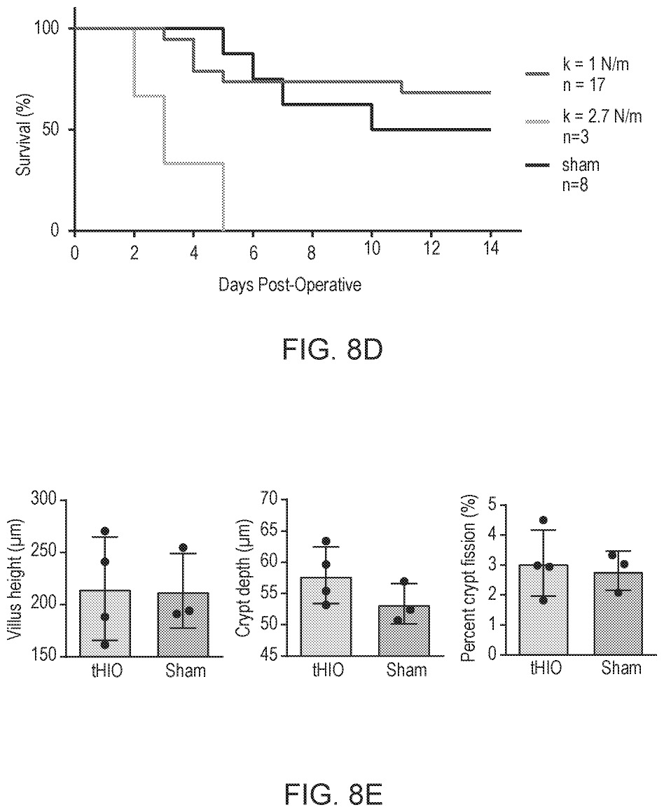

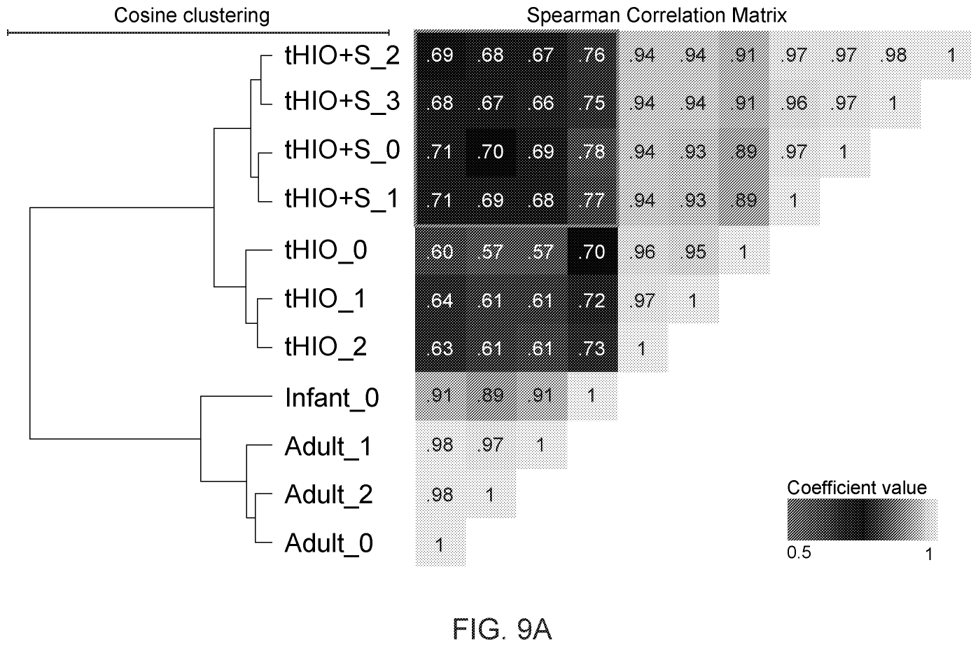

Applicant previously developed a method of stepwise growth factor manipulations to differentiate human pluripotent stem cells into intestinal organoids²⁻⁵. Upon transplantation into the mesentery of NOD-SCID IL-2Rγnull (NSG) mice these organoids indeed engraft and go on to closely resemble native intestine with well-defined crypt regions, villi, and smooth muscle layers⁴. After ten weeks, the tHIO has drastically grown in size. At this time, a secondary procedure was performed wherein a compressed spring was implanted inside the tHIO (FIG. 1A). 14 days (14 d) post implantation of the springs, the grafts were harvested and their human origins confirmed (FIG. 8A, 8B)¹⁹. The spring implantation first involves opening the tHIO, briefly flushing any accumulated mucous and inserting the encapsulated compressed spring into the luminal space of the tHIO before it is closed (FIG. 1B). Sham experiments of implanting empty capsules were also performed (FIG. 8C). The survival rates between sham and spring implanted tHIOs were similar (FIG. 8D). The spring's deployment could be monitored in vivo through microCT and was observed to be linear (FIG. 1C).

Use of the spring was based upon previous studies describing endoluminal lengthening devices^{14-17,20}. Applicant found the nickel-titanium (nitinol, NiTi) spring as an ideal device, because it had been successfully used in rats and did not require manipulation post implantation as hydraulic devices do^{14,17}. Another advantage in utilizing the NiTi spring is that a large amount of negative space was retained within the tHIO after insertion, which permitted mucous accumulation. The spring's geometry was based upon the constraint of a commercially available gelatin capsule used to maintain the spring in a compressed state during transplantation. An enteric coating of cellulose acetate phthalate (C-A-P) was used in order to delay the degradation of the gelatin capsule during implantation. The springs had a relaxed length of 12-13 mm, outer diameter of 2 mm and when encapsulated were 5-6 mm in length (FIG. 1D,E). The pitch of the spring was designed to achieve a spring constant of 1.05±0.11 N/m (FIG. 1F). Springs with higher constants deployed in a nonlinear fashion causing bowel obstructions and total mortality (FIG. 8d).

Morphological Maturation as a Result of Strain in the tHIO

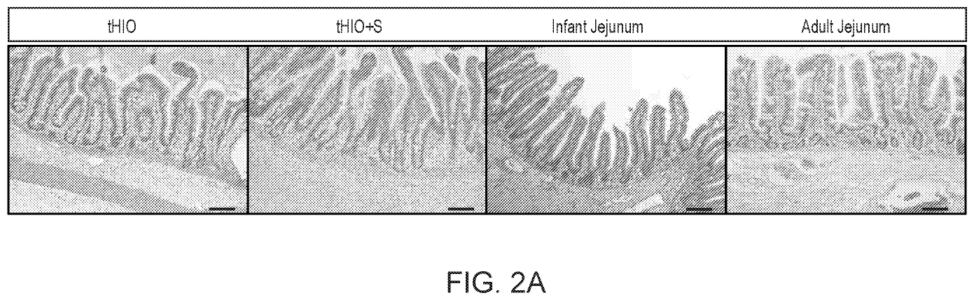

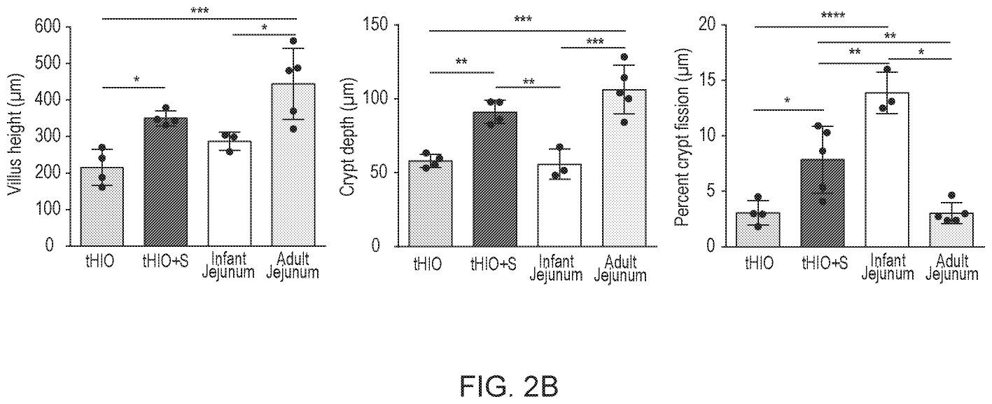

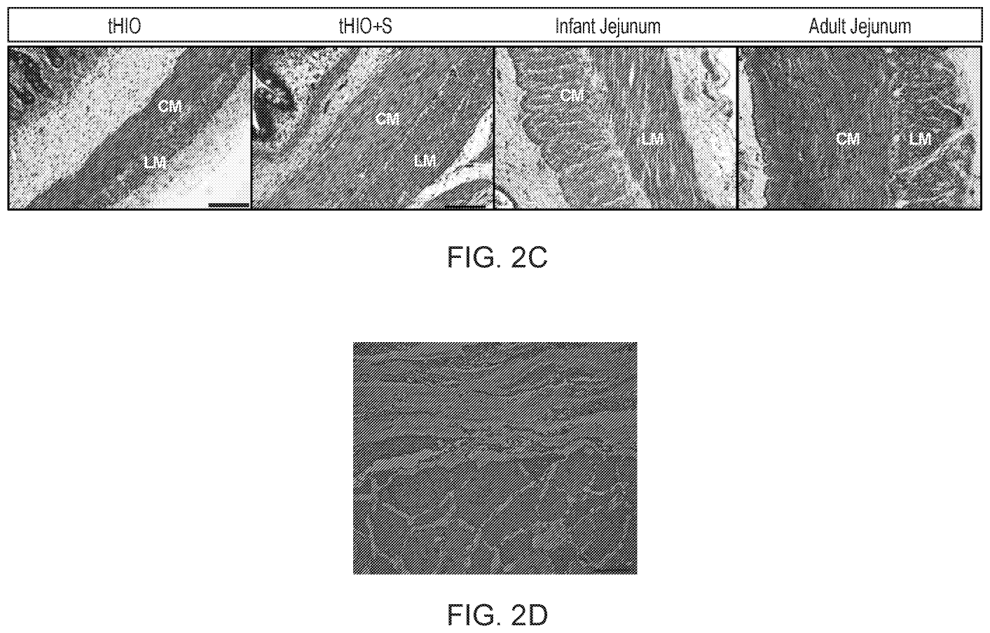

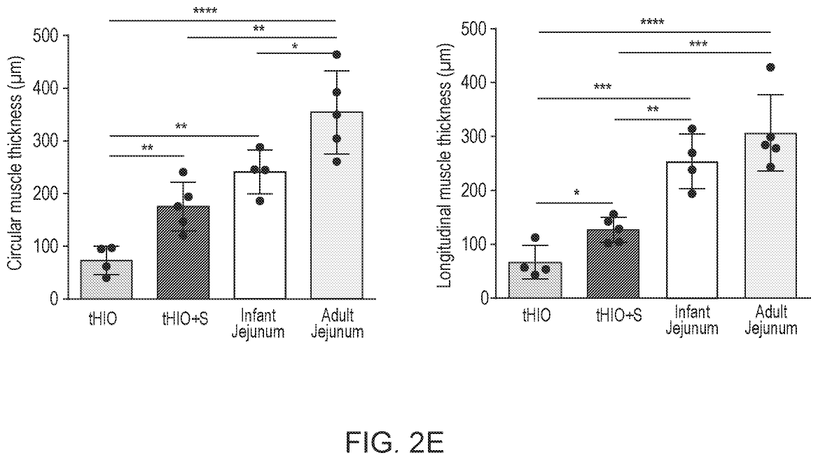

At the time of harvest, tHIO+S had a more tubular appearance verses the saccular shape of sham or tHIO and grew to about 10-14 mm (FIG. 8A). To examine if the application of strain produced an adaptive response, architectural features were quantified in tHIO, tHIO+S, and human surgical samples of infant and adult jejunum from hematoxylin and eosin stained sections (FIG. 2A). In tHIO+S, villus height, crypt depth and crypt fission were observed to be increased significantly over that of tHIO (FIG. 2B). The increase in both villus height and crypt depth approximated that of adult jejunum, while the level of crypt fission was higher in tHIO+S (FIG. 2B). Upon quantifying these features in sham operated grafts, no statistically significant differences were found when compared to tHIO indicating that the changes observed in tHIO+S were in association with strain and not surgically induced (FIG. 8D). All subsequent assays used the tHIO as a developmental control, as its attributes represent the current state of the field. Smooth muscle layers were also quantified across sample types using pentachrome stained sections (FIG. 2C). Perpendicular orientation of muscle fibers was observed in micrographs obtained using a transmission electron microscope (TEM) in both tHIO and tHIO+S (FIG. 2D, only tHIO+S shown). This observation confirmed the presence of circular and longitudinal muscle layers. The thicknesses of which were found to be increased significantly in tHIO+S over tHIO (FIG. 2E). While increased muscle thickness was observed in the tHIO+S, it did not yet approximate that of human adult jejunum. These data all suggest that a regulated tensile force improves the overall structure and geometry of tHIOs.

Strain's Impact on Transcriptional Changes in tHIOs.

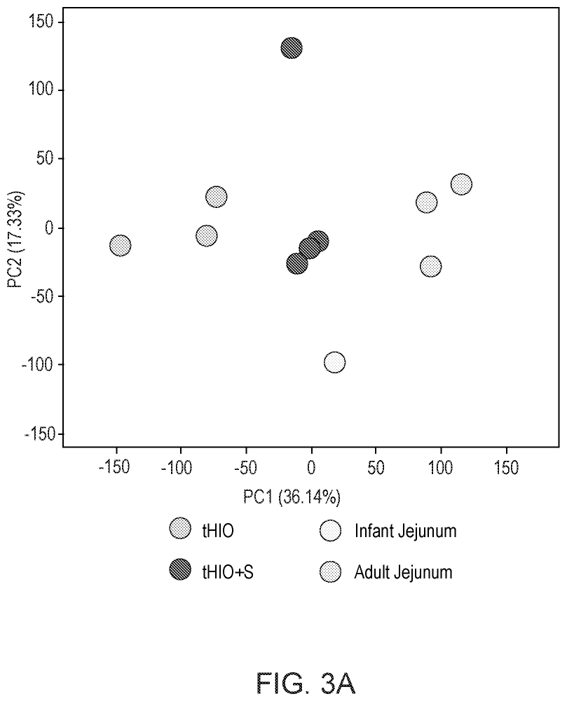

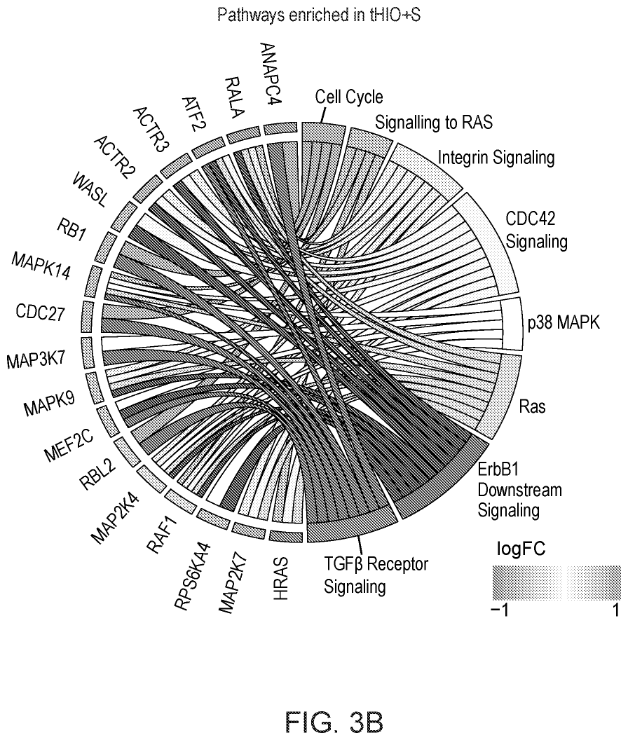

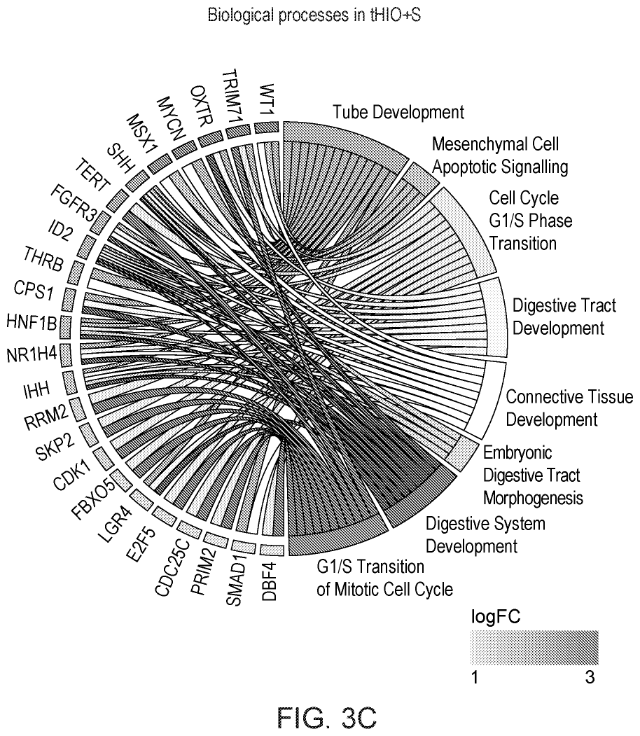

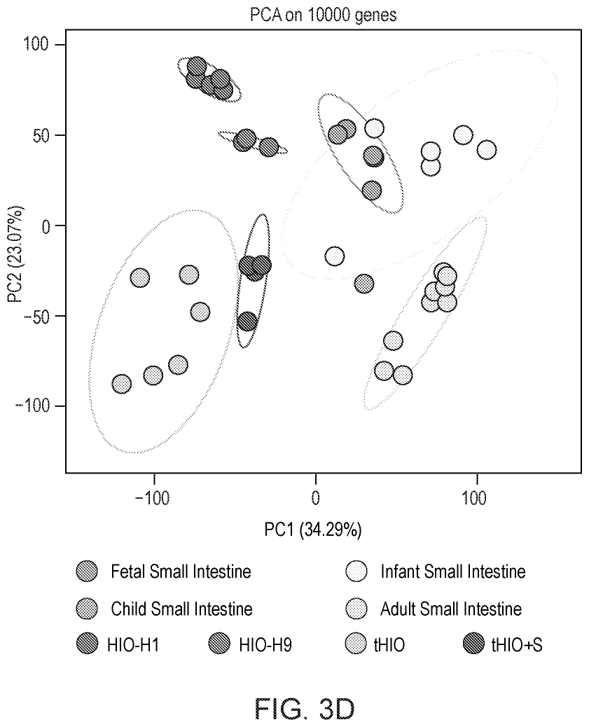

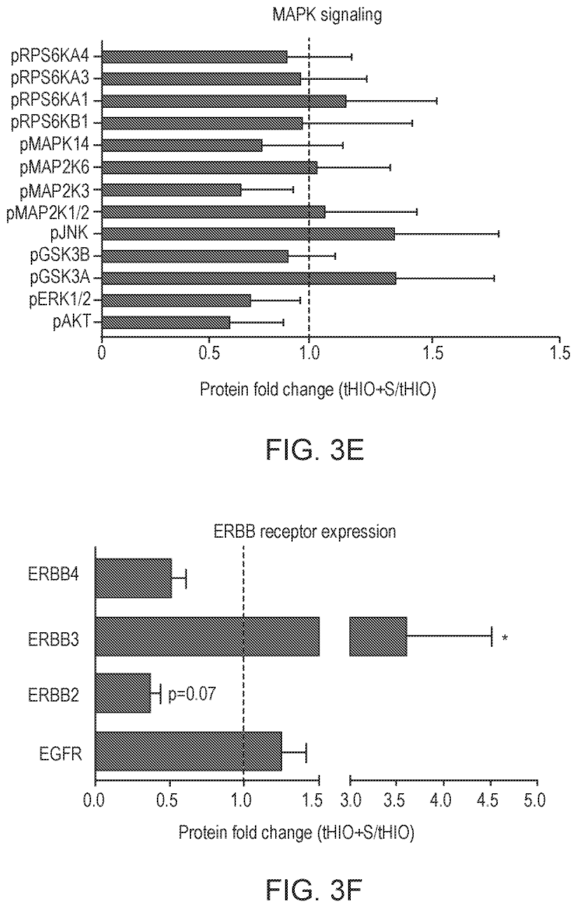

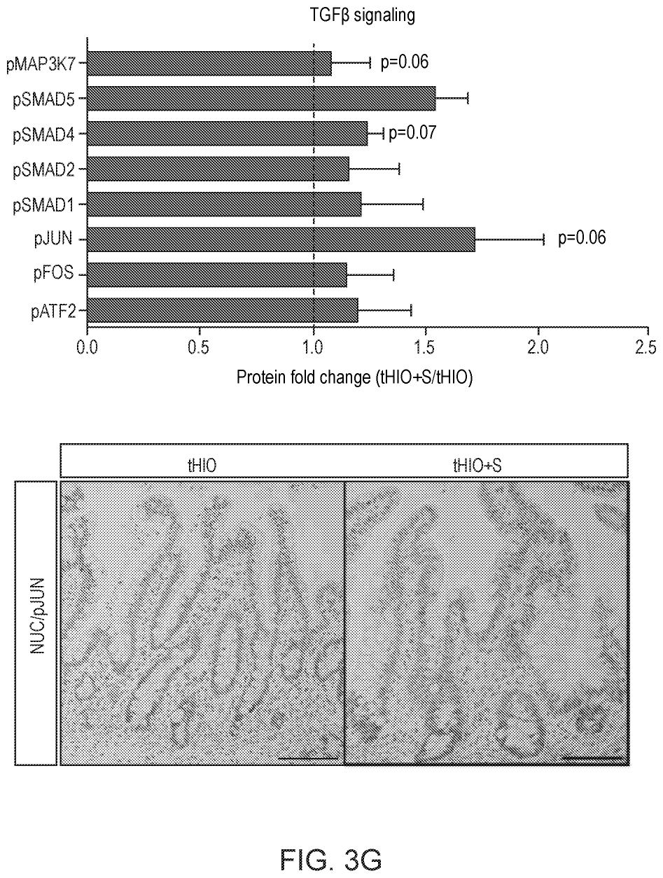

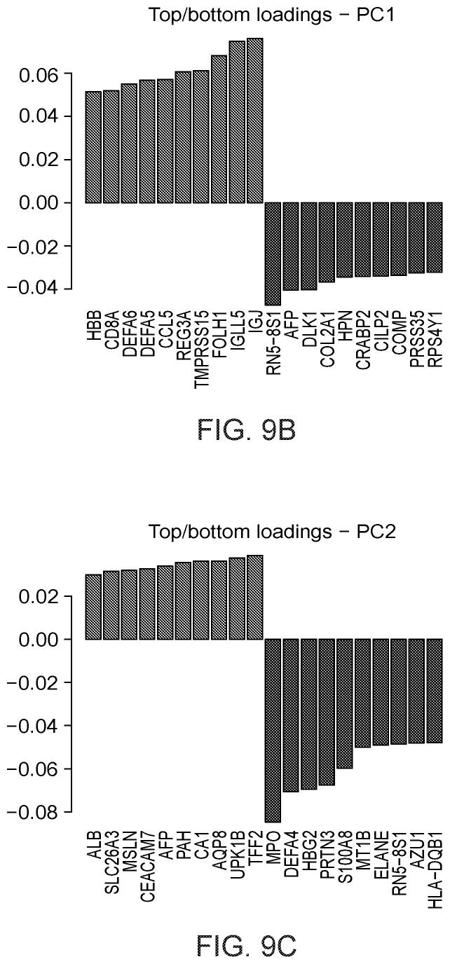

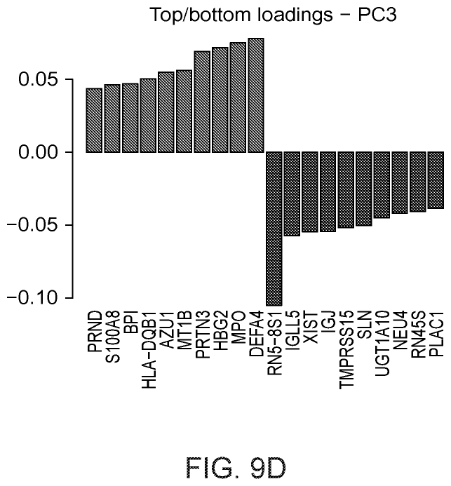

Applicant examined the differential gene expression in tHIO+S compared to tHIO and human jejunum (all full thickness samples) using RNA sequencing. Out of 23,366 genes annotated in the genome, 4,537 genes were significantly differentially regulated amongst the samples. Samples were analyzed using a scaled centered principle component analysis (PCA) to visualize the multi-dimensional variation between samples. Principal component 1 (36.14%), discriminated the samples among their types and suggested a higher degree of similarity between tHIO+S and human infant tissue (FIG. 3A). Unsupervised hierarchical clustering based on the Cosine distance and Spearman rank correlation, revealed similarities between tHIO+S and human infant tissues suggesting a pro-maturation effect of the applied strain (r²>0.75) (FIG. 9A). Applicant then performed a functional enrichment analysis on the PC loadings. Applicant found that PC1 segregated PSCs-derived tissues from Patient-derived tissues encompassing biological processes involving development (negative) and an immune signature (positive). PC2 segregated an overall intestinal signature from immune-competent intestinal tissues, encompassing immune and defense processes (negative) and digestion and metabolic processes (positive) (FIG. 9B, Table 1).

TABLE 1
Functional enrichment analysis of the principal component loadings
accounting for the biological processes seen in the PCA (FIG. 3A).
Functional enrichment for bottom loadings-PC1 (negative)

| Biological Process | Term | DE | P.DE |
|---|---|---|---|
| GO:0030198 | extracellular matrix organization | 59 | 4.38E−34 |
| GO:0043062 | extracellular structure organization | 59 | 5.29E−34 |
| GO:0048731 | system development | 215 | 1.02E−25 |
| GO:0048856 | anatomical structure development | 243 | 3.85E−25 |
| GO:0032502 | developmental process | 254 | 5.24E−25 |
| GO:0007275 | multicellular organism development | 230 | 6.10E−25 |
| GO:0044767 | single-organism developmental process | 250 | 2.60E−24 |
| GO:0009653 | anatomical structure morphogenesis | 141 | 5.01E−23 |
| GO:0044707 | single-multicellular organism process | 256 | 6.63E−23 |
| GO:0030574 | collagen catabolic process | 24 | 1.04E−21 |

TABLE 2
Functional enrichment for top loadings-PC1 (positive)

| Biological | | | |
|---|---|---|---|
| Process | Term | DE | P.DE |
| GO:0006955 | immune response | 197 | 4.10E−74 |
| GO:0002376 | immune system process | 232 | 4.58E−73 |
| GO:0002682 | regulation of immune | 151 | 2.65E−58 |
| | system process | | |
| GO:0002684 | positive regulation of | 123 | 1.70E−54 |
| | immune system process | | |
| GO:0045321 | leukocyte activation | 136 | 7.30E−54 |
| GO:0001775 | cell activation | 143 | 7.63E−53 |
| GO:0006952 | defense response | 151 | 3.48E−52 |
| GO:0046649 | lymphocyte activation | 101 | 5.82E−52 |
| GO:0050776 | regulation of immune | 115 | 8.93E−50 |
| | response | | |
| GO:0042110 | T cell activation | 78 | 6.74E−42 |

TABLE 3
Functional enrichment for bottom loadings-PC2 (negative)

| Biological Process | Term | DE | P.DE |
|---|---|---|---|
| GO:0002376 | immune system process | 156 | 5.12E−24 |
| GO:0006955 | immune response | 123 | 7.11E−23 |
| GO:0007155 | cell adhesion | 101 | 9.85E−23 |
| GO:0022610 | biological adhesion | 101 | 1.48E−22 |
| GO:0006952 | defense response | 102 | 4.85E−20 |
| GO:0044707 | single-multicellular organism process | 253 | 1.12E−19 |
| GO:0045321 | leukocyte activation | 87 | 1.85E−19 |
| GO:0001775 | cell activation | 92 | 1.31E−18 |
| GO:0002682 | regulation of immune system process | 91 | 1.36E−17 |
| GO:0050896 | response to stimulus | 305 | 1.71E−17 |

TABLE 4
Functional enrichment for top loadings-PC2 (positive)

| Biological Process | Term | DE | P.DE |
|---|---|---|---|
| GO:0007586 | digestion | 38 | 2.58E−25 |
| GO:0006811 | ion transport | 95 | 8.16E−17 |
| GO:0006629 | lipid metabolic process | 87 | 1.21E−16 |
| GO:0015711 | organic anion transport | 46 | 2.15E−16 |
| GO:0044281 | small molecule metabolic process | 110 | 9.75E−16 |
| GO:0006820 | anion transport | 51 | 6.11E−15 |
| GO:0022600 | digestive system process | 21 | 1.16E−14 |
| GO:0006641 | triglyceride metabolic process | 22 | 1.88E−14 |
| GO:0044710 | single-organism metabolic process | 172 | 2.18E−13 |
| GO:0006639 | acylglycerol metabolic process | 22 | 3.51E−13 |

TABLE 5
Functional enrichment for bottom loadings-PC3 (negative)

| Biological | | | |
|---|---|---|---|
| Process | Term | DE | P.DE |
| GO:0006614 | SRP-dependent cotranslation | 20 | 5.78E−14 |
| | prot. targeting to membrane | | |
| GO:0006613 | cotranslation protein targeting | 20 | 1.71E−13 |
| | to membrane | | |
| GO:0045047 | protein targeting to ER | 20 | 3.87E−13 |
| GO:0070972 | protein localization to | 22 | 4.74E−13 |
| | endoplasmic reticulum | | |
| GO:0072599 | protein localization to | 20 | 8.42E−13 |
| | endoplasmic reticulum | | |
| GO:0000184 | nuclear-transcribed mRNA | 20 | 1.15E−11 |
| | catabolic process | | |
| GO:0006413 | translation initiation | 22 | 1.23E−09 |
| GO:0019083 | viral transcription | 21 | 1.33E−09 |
| GO:0044270 | cellular nitrogen compound | 33 | 1.49E−09 |
| | catabolic process | | |
| GO:0006612 | protein targeting to membrane | 21 | 2.26E−09 |

TABLE 6
Functional enrichment for top loadings-PC3 (positive)

| Biological | | | |
|---|---|---|---|
| Process | Term | DE | P.DE |
| GO:0044707 | single-multicellular organism process | 256 | 2.29E−22 |
| GO:0032501 | multicellular organismal process | 273 | 8.34E−19 |
| GO:0044767 | single-organism developmental process | 226 | 7.29E−15 |
| GO:0032502 | developmental process | 228 | 9.51E−15 |
| GO:0048731 | system development | 188 | 9.78E−15 |
| GO:0048856 | anatomical structure development | 215 | 3.57E−14 |
| GO:0007275 | multicellular organism development | 202 | 5.84E−14 |
| GO:0051179 | localization | 230 | 8.16E−14 |
| GO:0045055 | regulated exocytosis | 57 | 1.18E−13 |
| GO:0051239 | regulation of multicellular | 126 | 7.64E−13 |
| | organismal process | | |

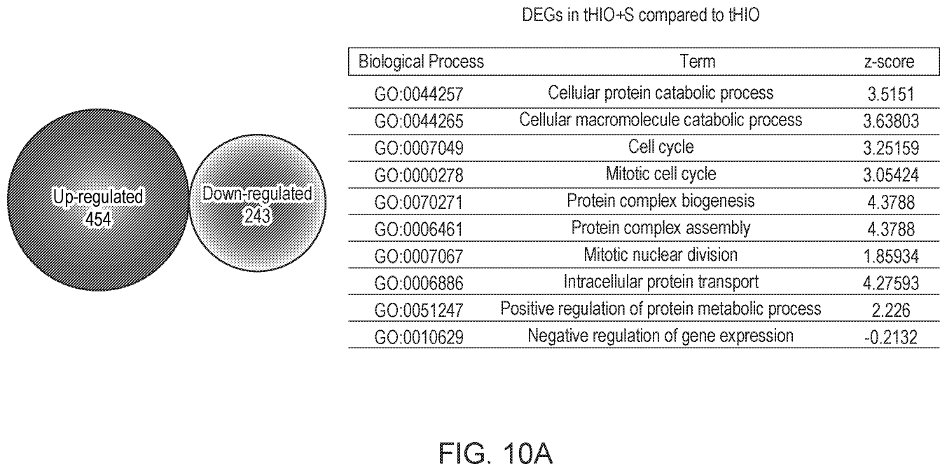

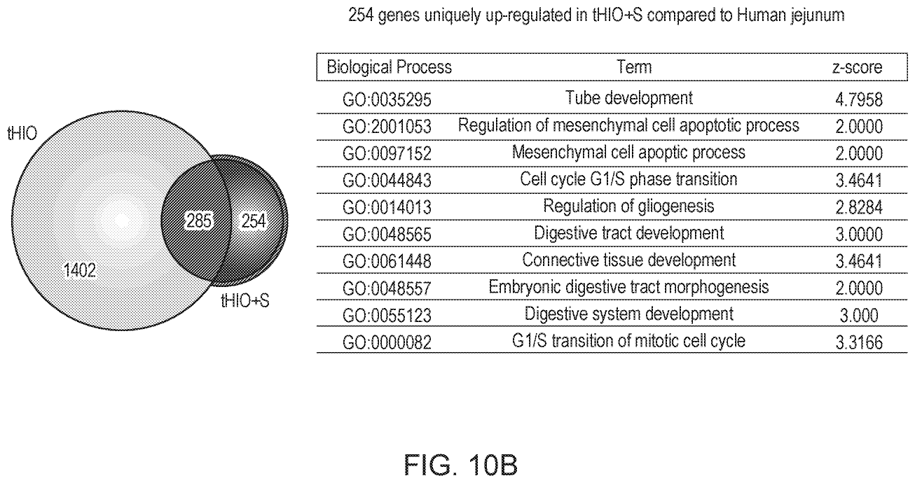

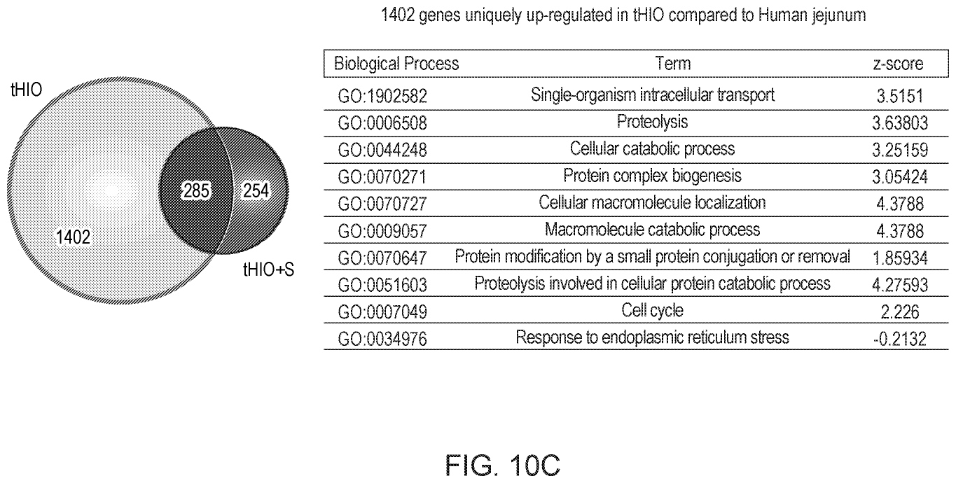

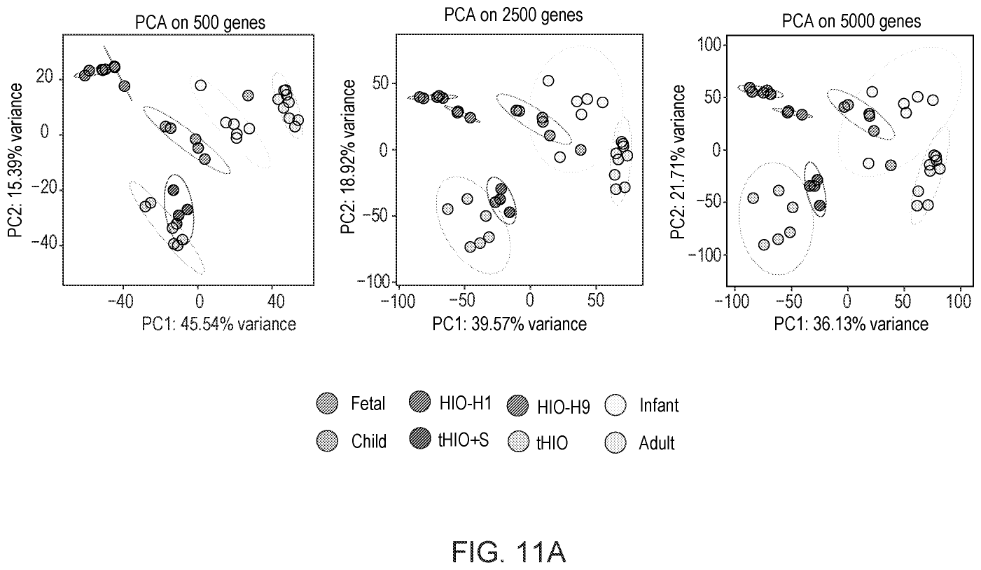

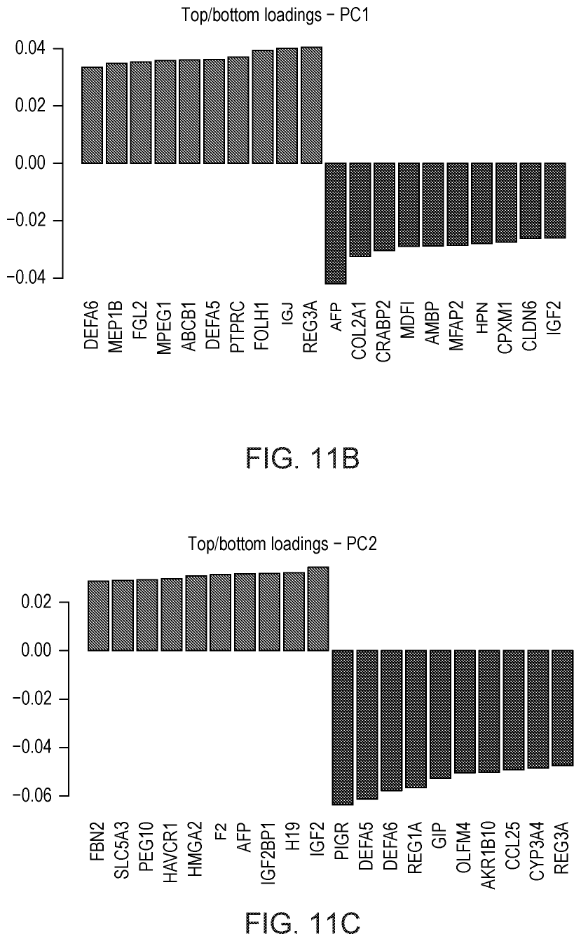

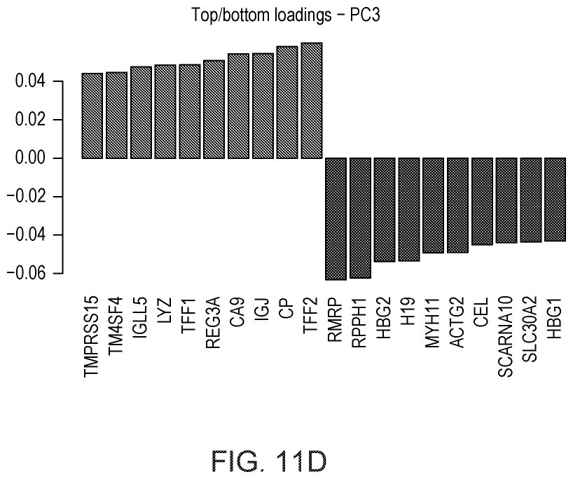

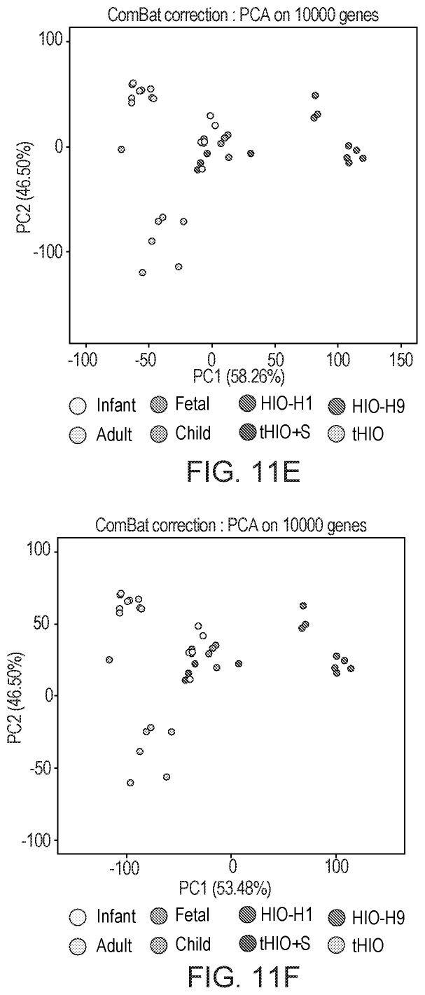

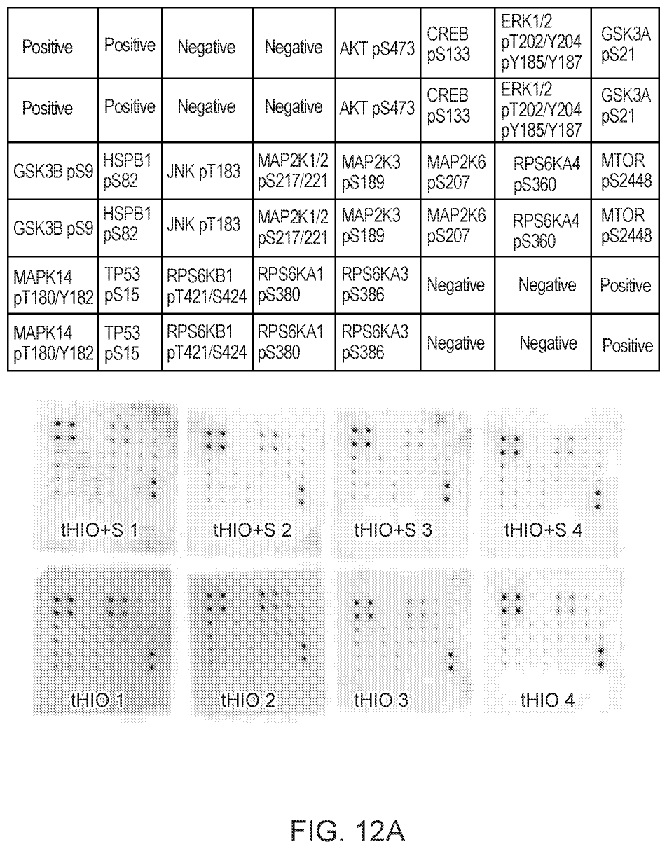

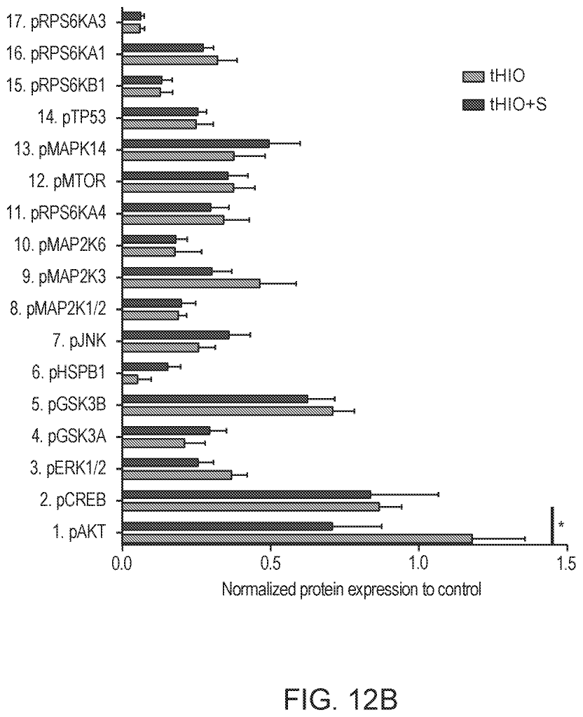

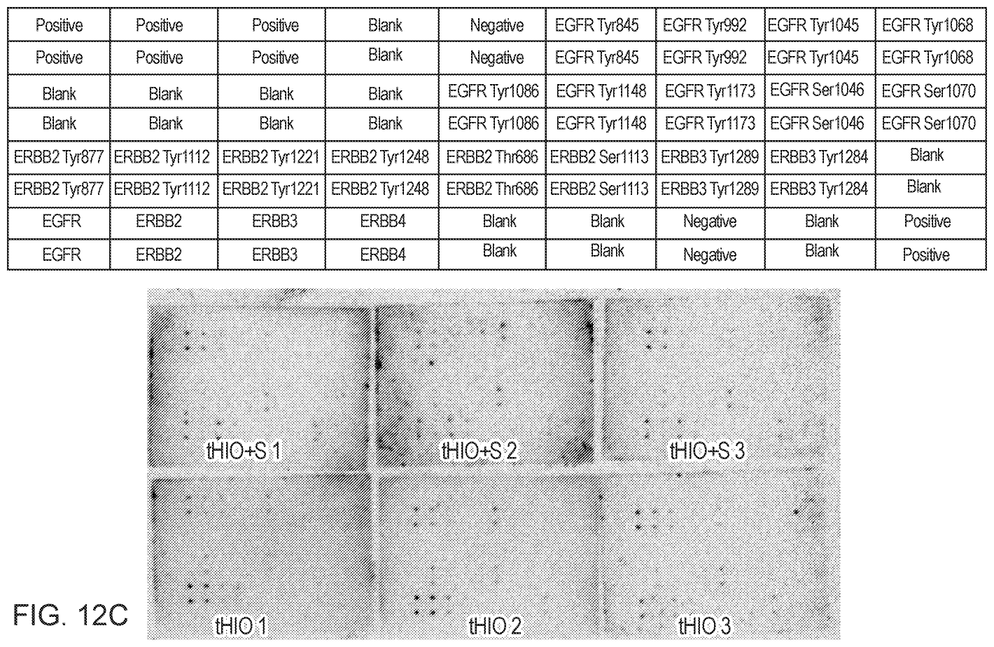

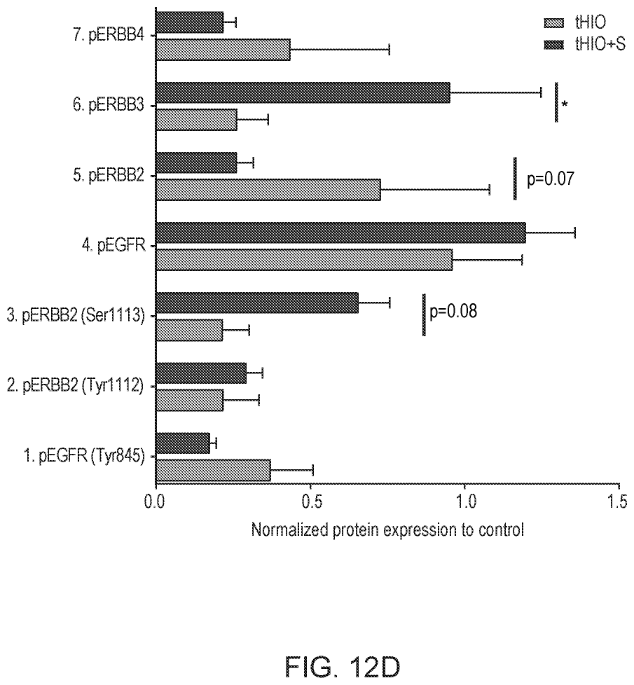

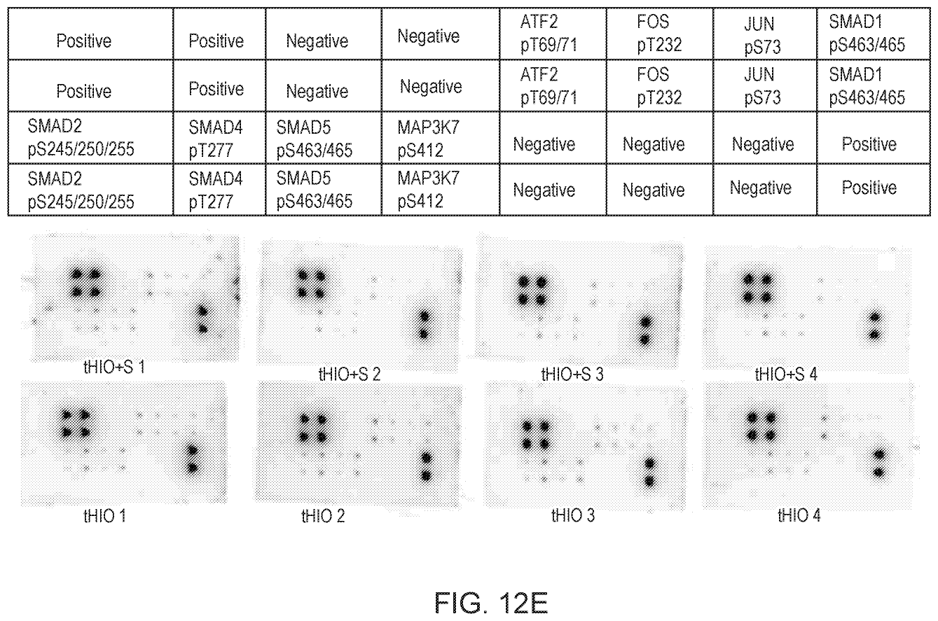

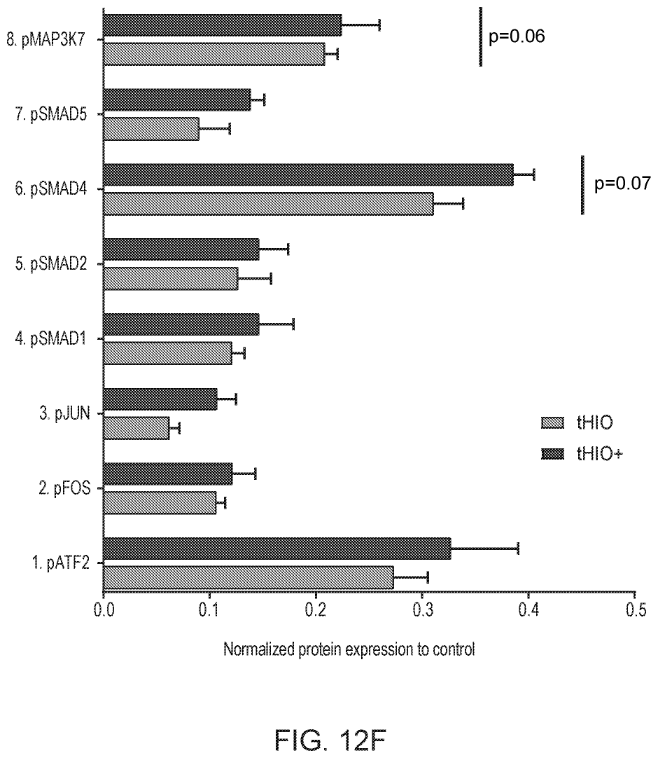

In order to examine the biological processes induced by strain in tHIO, Applicant compared the differentially expressed genes (DEGs) between the tHIO+S and tHIO groups. In tHIO+S compared to tHIOs, 454 and 243 genes were respectively up- and down-regulated. Next, a functional enrichment analysis was performed to highlight biological processes within these gene sets. 14 d after the initial application of strain implantation, cell cycle and overall catabolic and protein assembly processes were captured, suggesting an impact of strain upon cell proliferation and tissue formation (FIG. 10A). When further examining the gene ontology analysis of pathways enriched in tHIO+S compared to tHIO, significant pathways concerning the cell cycle, MAPK, ERBB and TGFβ signaling were observed. (FIG. 3B). Since the tHIO+S displayed a higher similarity with human jejunum tissues, Applicant compared their profiles. 254 genes were uniquely up-regulated in tHIO+S when compared to human jejunum tissues (FIG. 10B). Interestingly, the functional enrichment highlighted biological processes involving tube and digestive tract development, mesenchymal cell functions and cell cycle (FIG. 3C) that were not seen in tHIO when compared to human tissues (FIG. 10C). Applicant hypothesized that strain would enhance maturation of the tHIO. Applicant re-processed a collection of public RNAseq databases including fetal, newborn, human small intestinal tissues and HIO, tHIO and tHIO+S. A scaled centered PCA was performed to dimensionally cluster the samples. As previously demonstrated, the HIO clustered with fetal tissues. Interestingly, the tHIO+S were clustering toward human infant tissues thereby confirming the hypothesis (FIG. 3D; FIG. 11A). To gain additional insight from the principal components (PCs), Applicant performed a functional enrichment analysis on the PC loadings. Applicant found that PC1 segregated PSCs-derived tissues from Patient-derived tissues encompassing biological processes involving developmental processes and morphogenesis (negative) and an immune signature (positive). PC2 segregated fetal intestinal tissues from intestinal matured tissues, encompassing biological processes involving system development processes (positive) and digestion and metabolic processes (negative) (FIG. 12B-12E).

To broadly investigate some of the pathways enriched with exposure to strain beyond the level of transcription, protein analysis was performed. A protein phosphorylation array for MAPK signaling was executed and quantified (FIG. 3E). The fold changes in protein phosphorylation levels related to MAPK signaling were not consistently up or down across the panel, making it difficult to draw a conclusion as to its potential role. (For this and all subsequent protein analysis, array maps, developed membranes and normalized protein expression levels can be found in FIGS. 11A-11F and in the following Tables).

TABLE 7
Functional enrichment for bottom loadings-PC1 (negative) 2

| Biological Process | Term | DE | P.DE |
|---|---|---|---|
| GO:0032502 | developmental process | 236 | 9.43E−18 |
| GO:0048856 | anatomical structure development | 222 | 9.04E−17 |
| GO:0044767 | single-organism developmental process | 229 | 3.95E−16 |
| GO:0044707 | single-multicellular organism process | 235 | 5.04E−15 |
| GO:0048731 | system development | 188 | 5.93E−15 |
| GO:0007275 | multicellular organism development | 204 | 7.65E−15 |
| GO:0009653 | anatomical structure morphogenesis | 121 | 3.09E−14 |
| GO:0030198 | extracellular matrix organization | 36 | 5.69E−14 |
| GO:0043062 | extracellular structure organization | 36 | 6.29E−14 |
| GO:0001501 | skeletal system development | 43 | 1.48E−12 |

TABLE 8
Functional enrichment for top loadings-PC1 (positive)

| Biological | | | |
|---|---|---|---|
| Process | Term | DE | P.DE |
| GO:0006955 | immune response | 190 | 1.62E−72 |
| GO:0002376 | immune system process | 222 | 2.44E−70 |
| GO:0002682 | regulation of immune | 142 | 5.44E−54 |
| | system process | | |
| GO:0002684 | positive regulation of immune | 116 | 7.65E−51 |
| | system process | | |
| GO:0045321 | leukocyte activation | 129 | 8.11E−51 |
| GO:0050776 | regulation of immune response | 111 | 1.24E−48 |
| GO:0001775 | cell activation | 134 | 1.47E−48 |
| GO:0006952 | defense response | 140 | 8.90E−47 |
| GO:0046649 | lymphocyte activation | 89 | 9.65E−43 |
| GO:0050778 | positive regulation of | 84 | 6.64E−37 |
| | immune response | | |

TABLE 9
Functional enrichment for bottom loadings-PC2 (negative)

| Biological | | | |
|---|---|---|---|
| Process | Term | DE | P.DE |
| GO:0007586 | digestion | 38 | 1.23E−25 |
| GO:0044281 | small molecule metabolic process | 124 | 4.29E−23 |
| GO:0044710 | single-organism metabolic process | 181 | 1.47E−17 |
| GO:0006629 | lipid metabolic process | 87 | 3.28E−17 |
| GO:0006805 | xenobiotic metabolic process | 24 | 5.57E−17 |
| GO:0006082 | organic acid metabolic process | 75 | 5.69E−17 |
| GO:0071466 | cellular response to xenobiotic | 24 | 1.44E−16 |
| | stimulus | | |
| GO:0043436 | oxoacid metabolic process | 73 | 3.07E−16 |
| GO:0019752 | carboxylic acid metabolic process | 68 | 5.51E−16 |
| GO:0009410 | response to xenobiotic stimulus | 24 | 5.53E−16 |

TABLE 10
Functional enrichment for top loadings-PC2 (positive)

| Biological | | | |
|---|---|---|---|
| Process | Term | DE | P.DE |
| GO:0044707 | single-multicellular | 200 | 2.23E−11 |
| | organism process | | |
| GO:0048856 | anatomical structure | 180 | 3.66E−10 |
| | development | | |
| GO:0007275 | multicellular organism | 168 | 8.67E−10 |
| | development | | |
| GO:0044767 | single-organism developmental | 186 | 1.07E−09 |
| | process | | |
| GO:0032502 | developmental process | 188 | 1.10E−09 |
| GO:0048731 | system development | 152 | 3.01E−09 |
| GO:0032501 | multicellular organismal process | 213 | 1.13E−08 |
| GO:0051960 | regulation of nervous | 43 | 1.39E−08 |
| | system development | | |
| GO:0050919 | negative chemotaxis | 8 | 1.89E−08 |
| GO:0007399 | nervous system development | 88 | 3.17E−08 |

TABLE 11
Functional enrichment for bottom loadings-PC3 (negative)

| Biological Process | Term | DE | P.DE |
|---|---|---|---|
| GO:0006334 | nucleosome assembly | 30 | 4.00E−20 |
| GO:0031497 | chromatin assembly | 30 | 1.18E−18 |
| GO:0006342 | chromatin silencing | 26 | 8.39E−18 |
| GO:0006335 | DNA replication-dependent nucleosome assembly | 16 | 9.16E−18 |
| GO:0034723 | DNA replication-dependent nucleosome organization | 16 | 9.16E−18 |
| GO:0034728 | nucleosome organization | 30 | 9.43E−18 |
| GO:0006333 | chromatin assembly or disassembly | 31 | 1.19E−17 |
| GO:0045814 | negative regulation of gene expression, epigenetic | 26 | 2.56E−16 |
| GO:0000183 | chromatin silencing at rDNA | 16 | 4.88E−16 |
| GO:0006323 | DNA packaging | 30 | 5.86E−16 |

TABLE 12
Functional enrichment for top loadings-PC3 (positive)

| Biological | | | |
|---|---|---|---|
| Process | Term | DE | P.DE |
| GO:0007586 | digestion | 24 | 1.04E−11 |
| GO:0051179 | localization | 215 | 1.63E−10 |
| GO:0044710 | single-organism | 163 | 2.89E−10 |
| | metabolic process | | |
| GO:0044281 | small molecule | 96 | 4.63E−10 |
| | metabolic process | | |
| GO:0050896 | response to stimulus | 270 | 9.19E−10 |
| GO:0044283 | small molecule | 39 | 9.56E−10 |
| | biosynthetic process | | |
| GO:0002376 | immune system process | 115 | 2.04E−09 |
| GO:1901615 | organic hydroxy compound | 36 | 3.94E−09 |
| | metabolic process | | |
| GO:0045047 | protein targeting to ER | 16 | 6.19E−09 |
| GO:0061621 | canonical glycolysis | 9 | 7.16E−09 |

Pan ERBB receptor expression was also measured and quantified (FIG. 3F). ERBB3 was found to be significantly increased in tHIO+S over tHIO. Finally, TGFβ signaling was investigated; a protein phosphorylation array was performed and quantified (FIG. 3G, left panel). While, statistical significance was not found due to high variability, there did appear to be an overall increasing trend in TGFβ pathway activation as measured by phosphorylation. The most striking difference was found to be in phosphorylated Jun Proto-Oncogene, AP-1 Transcription Factor Subunit (pJUN), which was further validated through immunohistochemistry (FIG. 3G, right panel). In staining for pJUN, an obvious visual increase was observed. With strain, pJUN expression expanded along the entirety of the villus rather than being predominantly expressed in the crypt with low level expression towards the tips of the villi without strain exposure. This expansion of pJUN provides support for the earlier pathway analysis.

Strain's Impact on Proliferation and the Stem Compartment in tHIOs

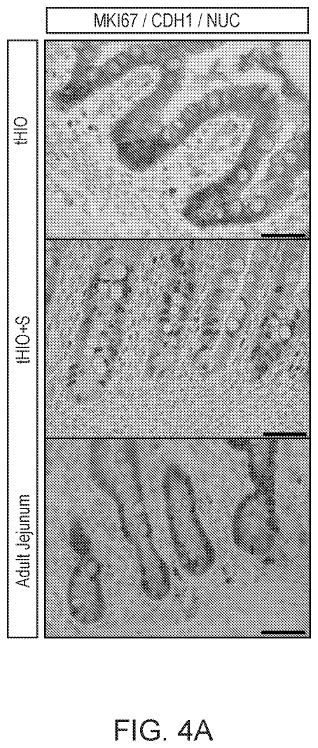

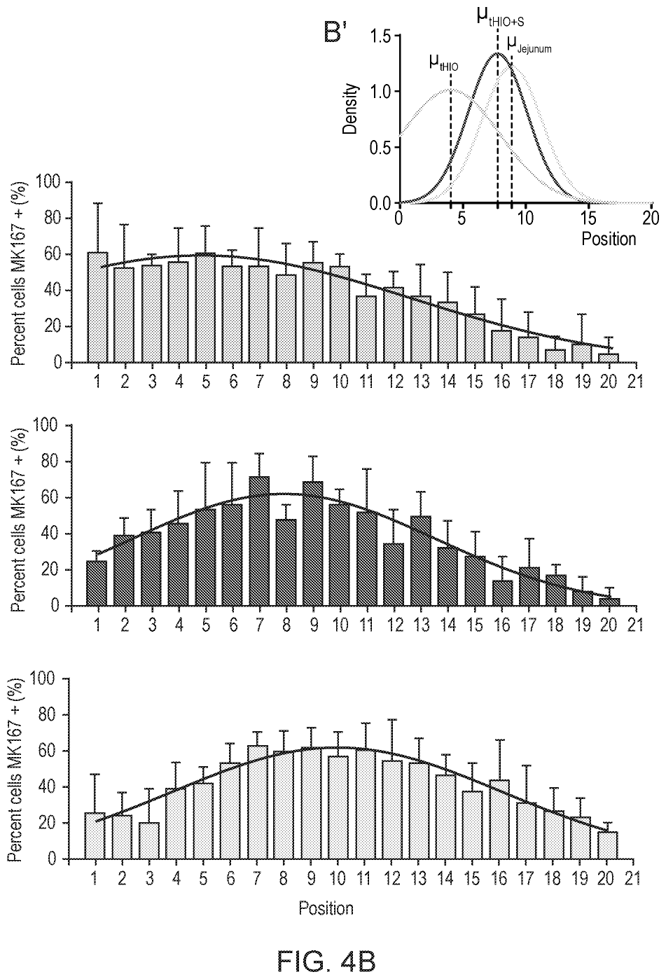

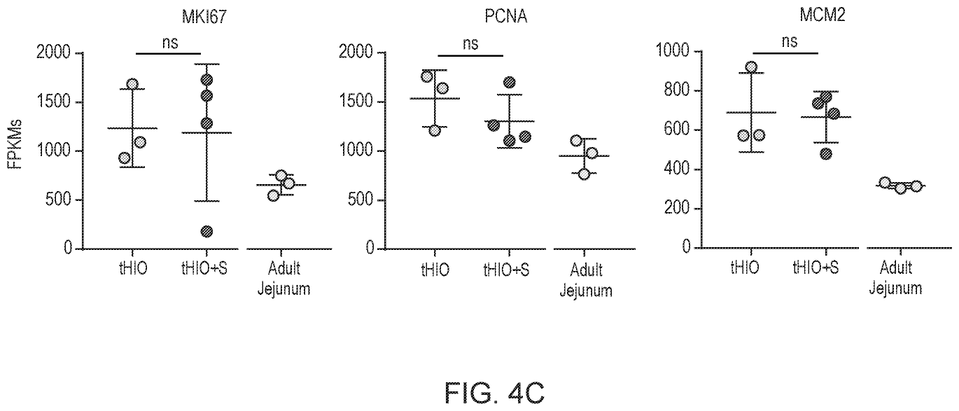

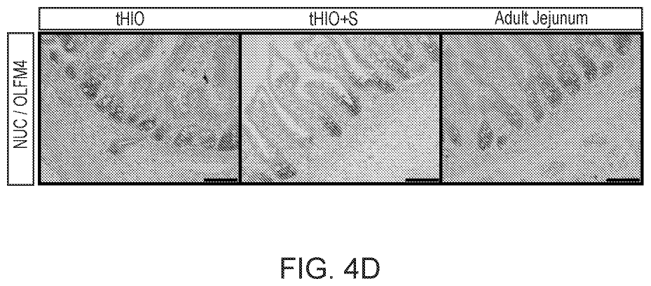

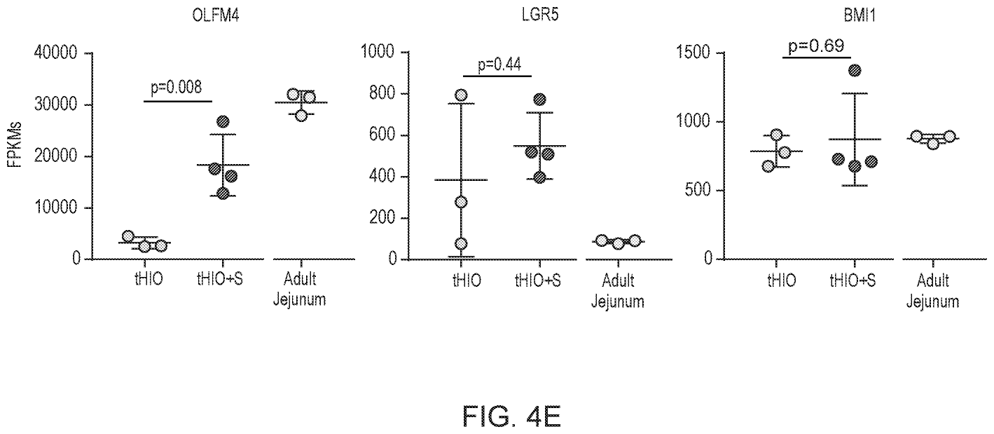

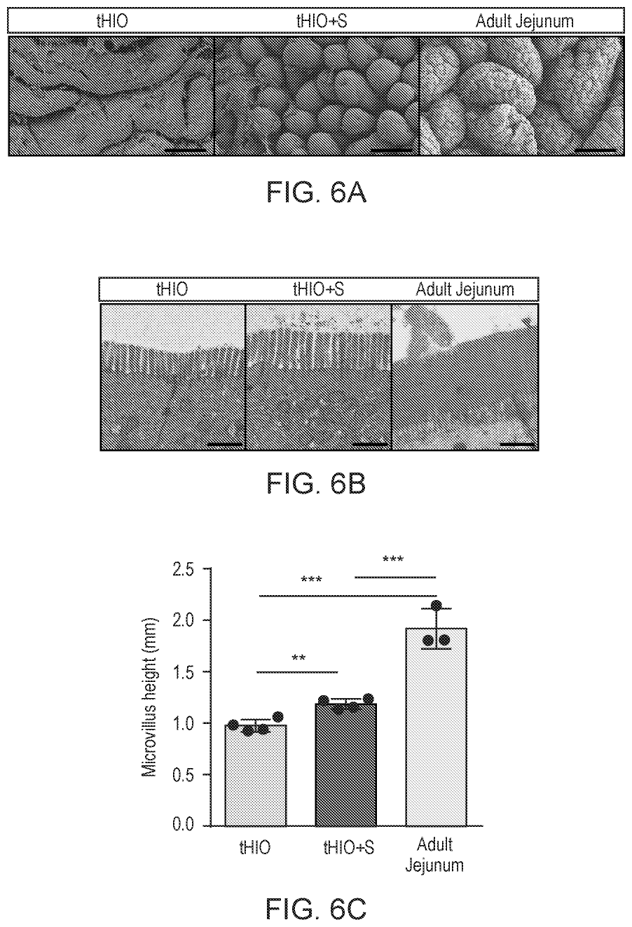

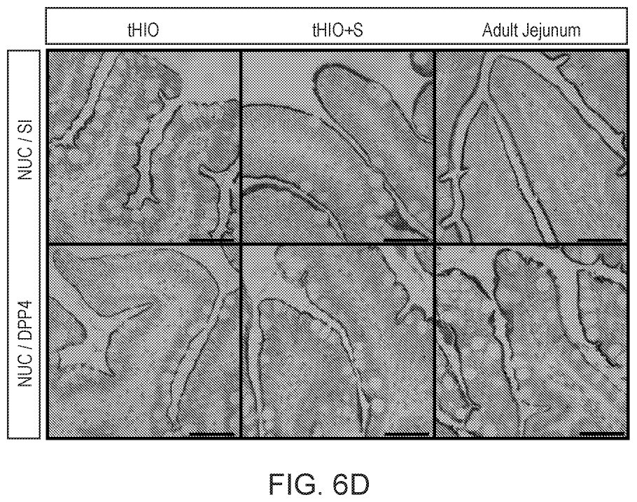

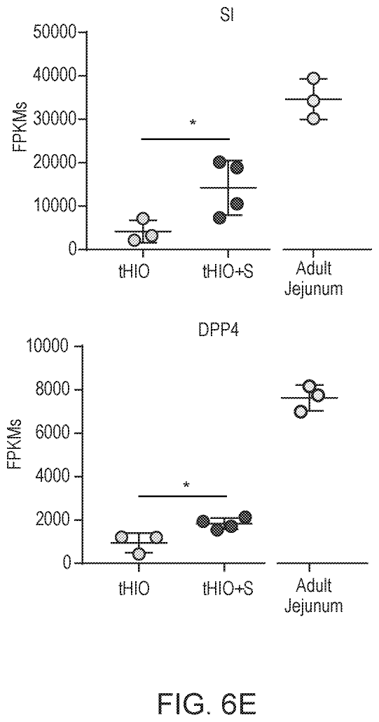

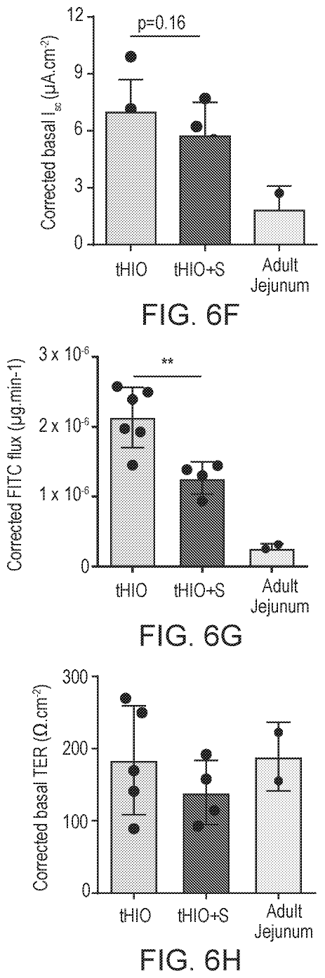

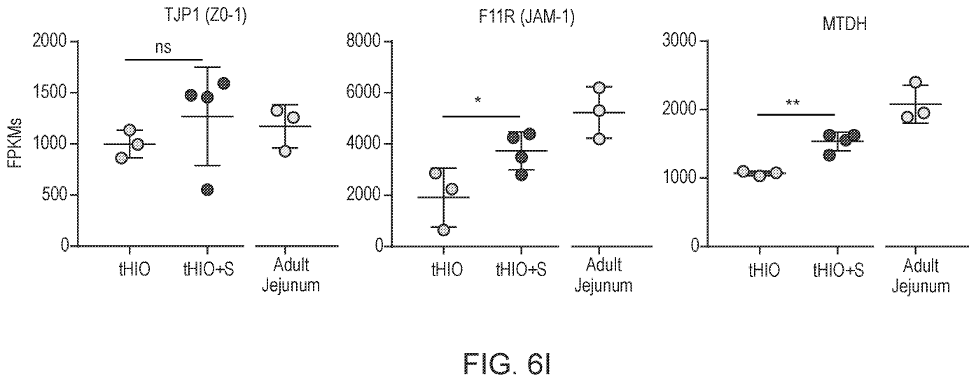

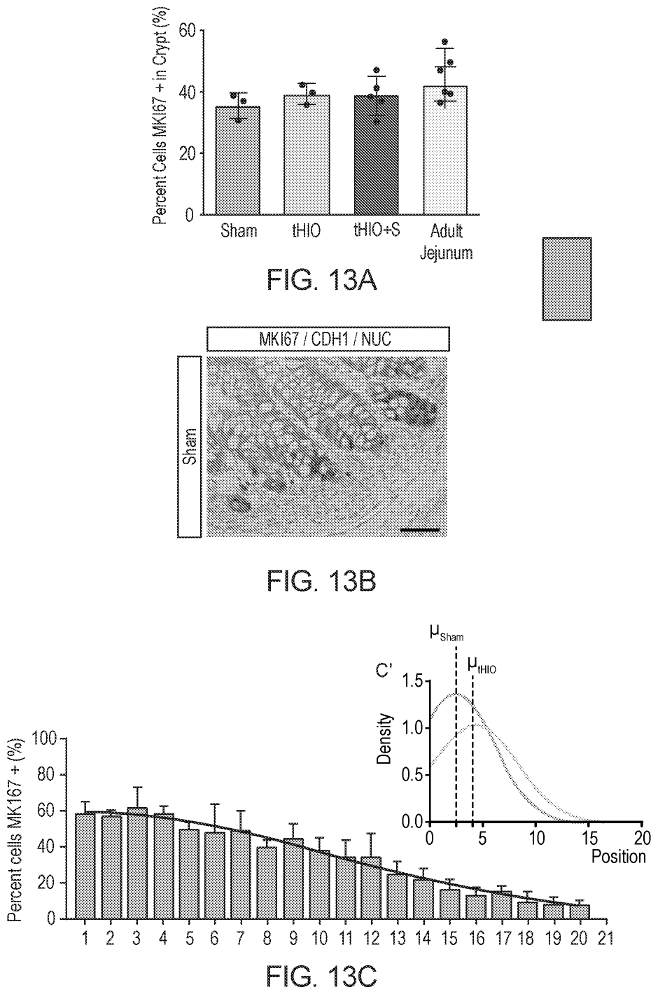

To examine whether strain could impact the intestinal stem cell/transit-amplifying compartment, Applicant analyzed proliferation in the crypts of tHIOs and tHIO+S and human adult jejunum. Dual chromogenic staining for Marker of Proliferation K167 (MK167) and Cadherin-1 (CDH1) was performed for the purpose of quantification (FIG. 4A). Total epithelial proliferation within the crypt was similar between groups (FIG. 6A). However, when comparing the means of Gaussian curve fits of the proliferation profiles by position, the tHIO+S exhibited an upward shift within the crypt compartment, which followed a pattern that was similar to that observed in adult jejunum (FIG. 4A-B′). This suggests the development of a transient amplifying zone within tHIO+S. The amount of proliferation in sham operated tissues was also quantified and found to follow a similar pattern to that of the tHIO (FIG. 13B-13C′). In total tissue, transcripts for the cell cycle related genes MKI67, Proliferating Cell Nuclear Antigen (PCNA) and Minichromosome Maintenance Complex Component 2 (MCM2) were observed as similar between tHIO+S and tHIO, while both were elevated compared to adult jejunum. To further examine the intestinal stem cell compartment, staining for Olfactomedin 4 (OLFM4) was performed and all sample types displayed strong positivity exclusive to the crypt (FIG. 4D). Transcripts for OLFM4 were found to be significantly increased in tHIO+S compared to tHIO, while additional intestinal stem cell markers Leucine-rich repeat-containing G-protein coupled receptor 5 (LGR5) and BMI1 Proto-Oncogene, Polycomb Ring Finger (BMI1) were observed to be similar between the two groups.

Epithelial Response of tHIOs to Strain

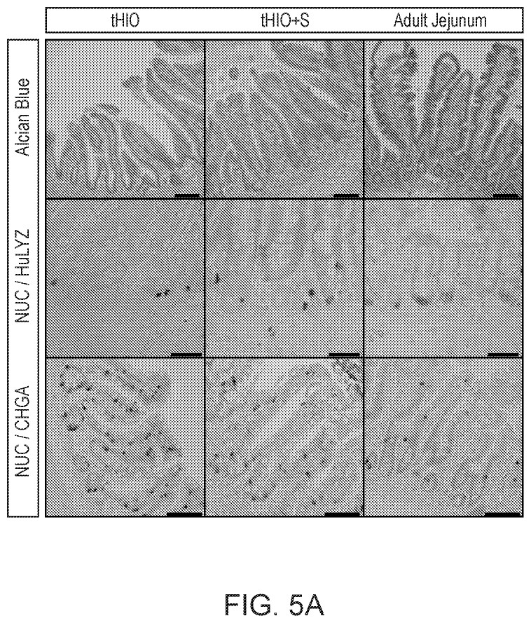

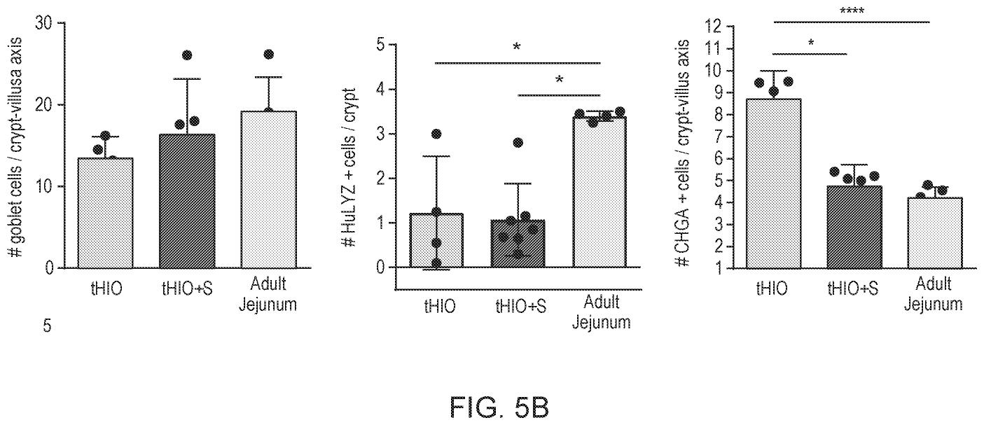

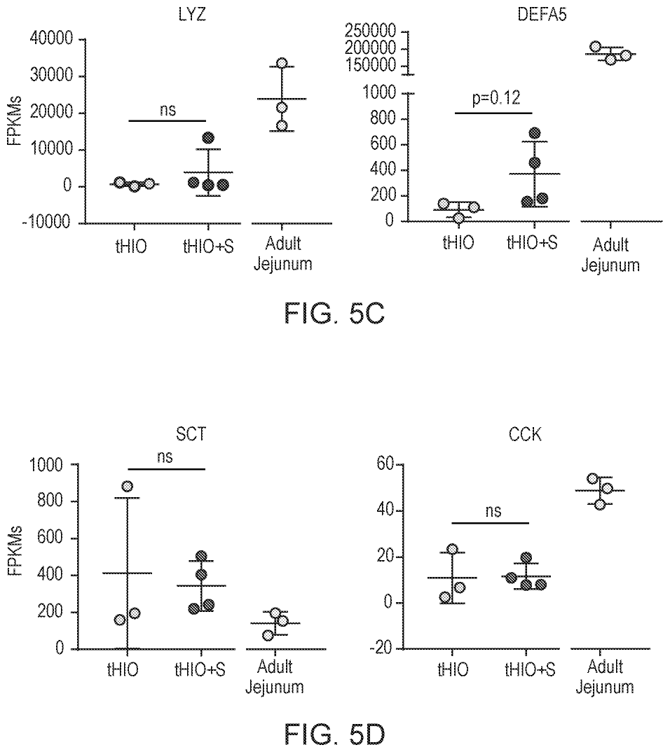

To evaluate if strain impacted cell fate, secretory epithelial cell types were quantified. Alcian blue staining was performed for the identification of Goblet cells, human specific Lysozyme (LYZ) for Paneth cells and Chromogranin-a (CHGA) for enteroendocrine cells (EECs) which were all subsequently quantified (FIG. 5A, 5B). No difference in goblet cell counts were observed across sample types. The number of Paneth cells per crypt was also found similar between tHIO+S and tHIO, yet significantly lower than that of adult jejunum. However, when looking at antimicrobial peptide alpha-Defensin 5 (DEFA) transcription levels, an increasing trend was observed while those for LYZ remained unchanged (FIG. 5C). The number of CHGA+ EECs was significantly reduced in tHIO+S compared to tHIO and approximated that of adult jejunum. Interestingly, the transcripts for EEC secreted hormones Serotonin (SCT) and Cholecystokinin (CCK) were not reduced. Therefore, this data may suggest strain can negatively regulate EEC specification or that they serve as a progenitor pool. Taken together, the overall impact of strain on the developing graft's epithelium appears to be one of functional promotion and cell type normalization.

Epithelial topography of tHIO, tHIO+S, and adult jejunum samples was observed to see if strain impacted features of the surface ultrastructure (FIG. 6A). These scanning electron micrographs depicted ridge-like protrusions in the tHIO. while long finger-like villus projections were found in tHIO+S. This increase in structuration suggests that strain indeed promoted the villification process, as previously described¹¹. Deep corrugations in PSC generated tissues were quite rare, but found to be a prominent feature of the adult jejunum's large finger-like villi. This additional epithelial folding drastically increases the tissue's surface area increasing the gut's efficiency in digestive processes²¹. These observations suggest strain improved the architectural features of the tHIO, while further complexities, like corrugations, remain largely underdeveloped.

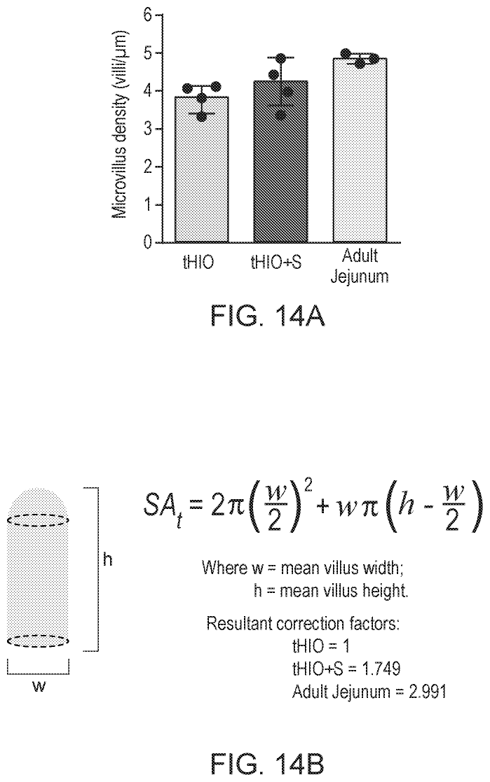

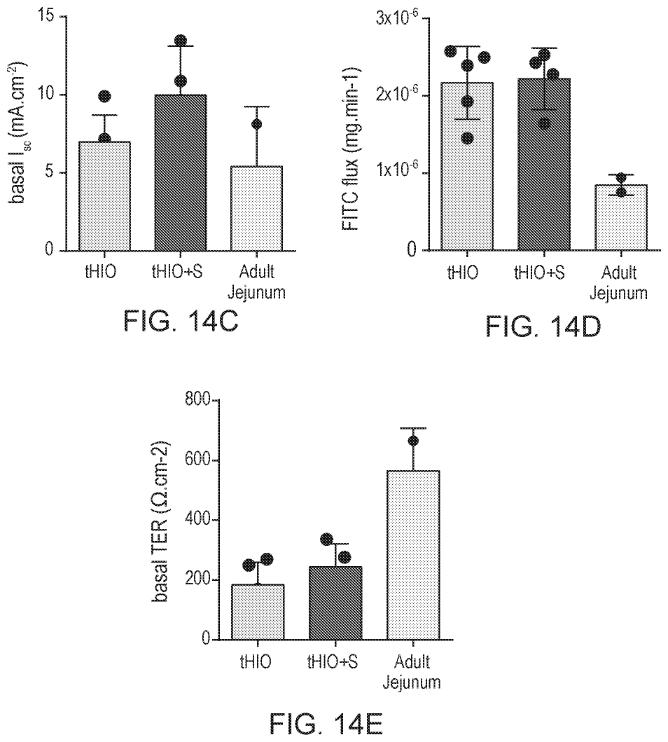

In addition to larger scale structural features, Applicant also wanted to examine aspects related to epithelial function to see if strain had modulated them. On top of overall villus length, the brush border's size impacts the available surface area for nutrient exchange within the gut²². TEM micrographs depict the brush border in tHIO, tHIO+S and adult jejunum (FIG. 6B). Individual microvilli were quantified and a significant increase was seen in their length in tHIO+S verses tHIO (FIG. 6C). While an increase was observed, the microvilli in adult jejunum were much longer than those in tHIO+S (FIG. 6C). When quantifying the density of microvilli per unit length, an increasing trend was observed; however differences were not significant between groups (FIG. 14A). When performing immunohistochemistry for the brush border enzymes Sucrase-isomaltase (SI) and Dipeptidyl Peptidase-4 (DPP4), a strong presence was observed across all sample types (FIG. 6D). Transcriptionally, a significant increase in expression levels of SI and DPP4 was observed in tHIO+S compared to tHIO indicating the capacity for carbohydrate digestion of the tHIO may be increased with exposure to strain (FIG. 6E).

An Ussing chamber was used to measure the epithelial characteristics of short circuit current, fluorescein isothiocyanate (FITC)-dextran flux and transepithelial resistance to observe any functional changes related to strain. All values were corrected using a correction factor calculated from morphometric observations (FIG. 10B). Due to the differences in villus height between sample types a correction factor was applied to further normalize the available surface area of the samples while in the Ussing chamber. High variability was observed in short circuit measurements and there was not a significant difference between tHIO+S and tHIO (FIG. 6F; uncorrected in FIG. 14C). However, a trend toward adult jejunum was observed. This measure indicated that the level of active ion transport may not be heavily influenced by strain²³. FITC-dextran flux or permeability was also observed and found to be significantly decreased in tHIO+S compared to tHIO, again moving toward the levels observed in adult jejunum (FIG. 6G; uncorrected in FIG. 14D). This indicates less paracellular permeability and increased barrier function. Levels of transepithelial resistance (TER), an indicator of tight junction establishment²³, displayed moderate variability and was similar between groups (FIG. 6H; uncorrected in FIG. 14E). Transcripts for tight junction components Tight Junction Protein 1 (TJP1/ZO-1), F11 Receptor (F11R/JAM-1), and Metadherin (MTDH) were all measured (FIG. 6I). Significant increases in transcripts for F11R and MTDH were observed in tHIO+S compared to tHIO, while there was no difference in TJP1 expression levels between groups. Together these data suggest a positive impact of strain on barrier function. While in some cases the effect of strain on the epithelium was slight, some improvements were seen and there was no evidence of epithelial disruption in association with strain.

Ex Vivo Muscle Function in Response to Strain in tHIO

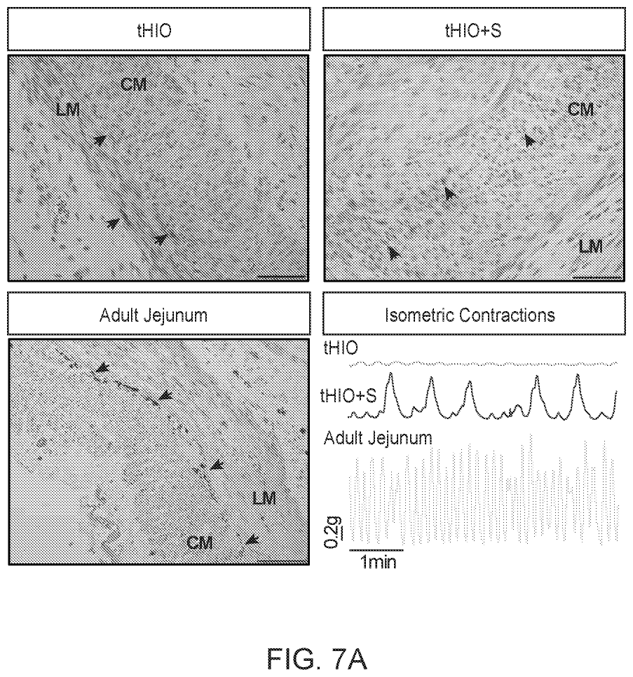

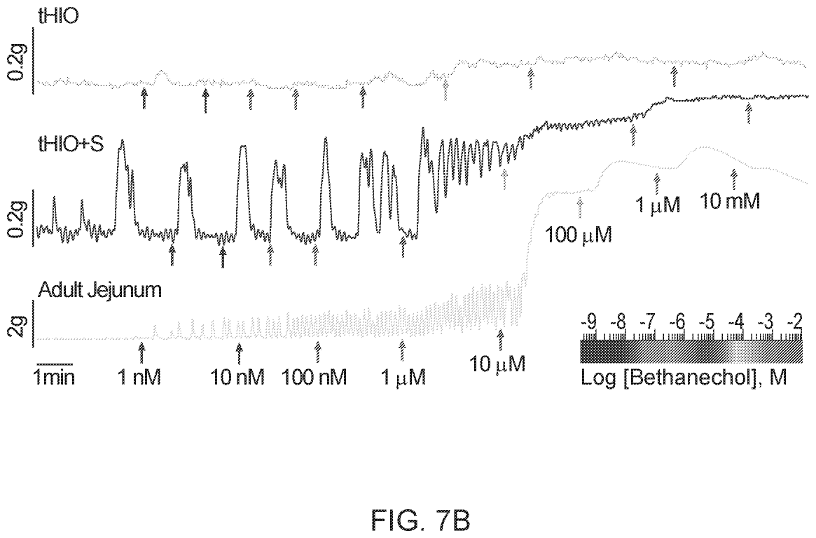

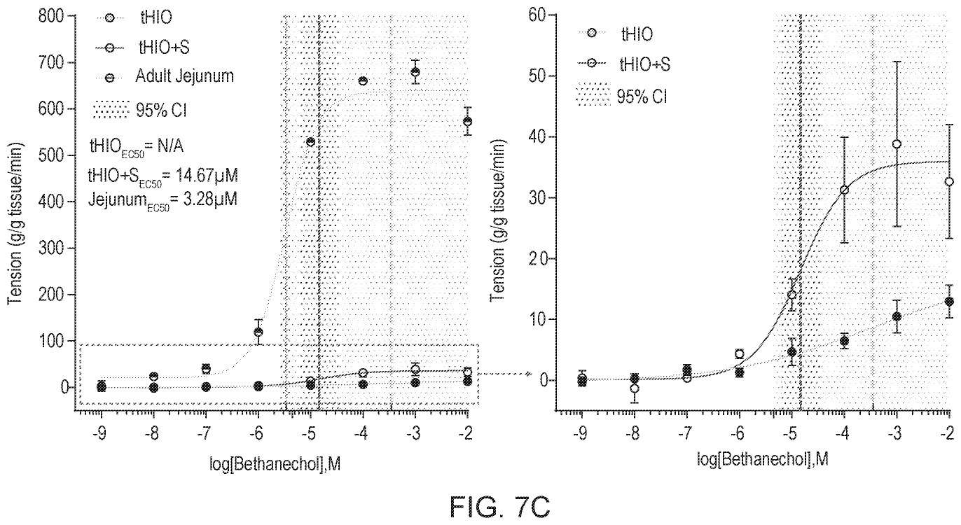

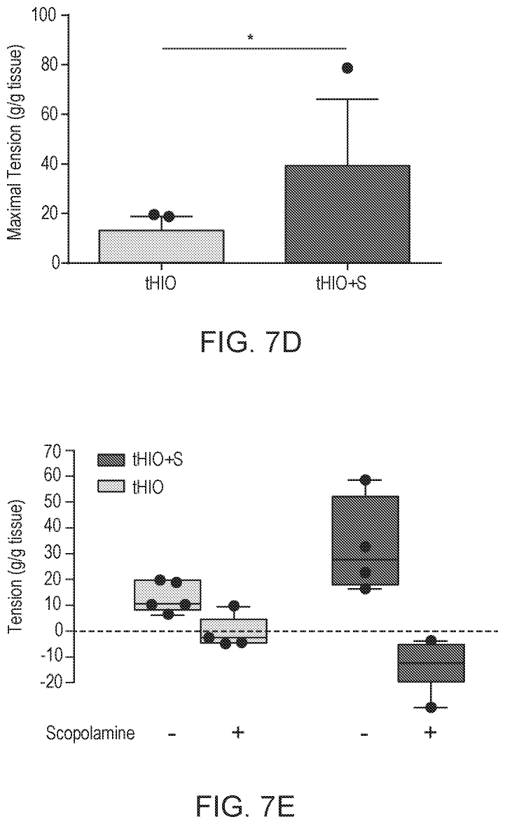

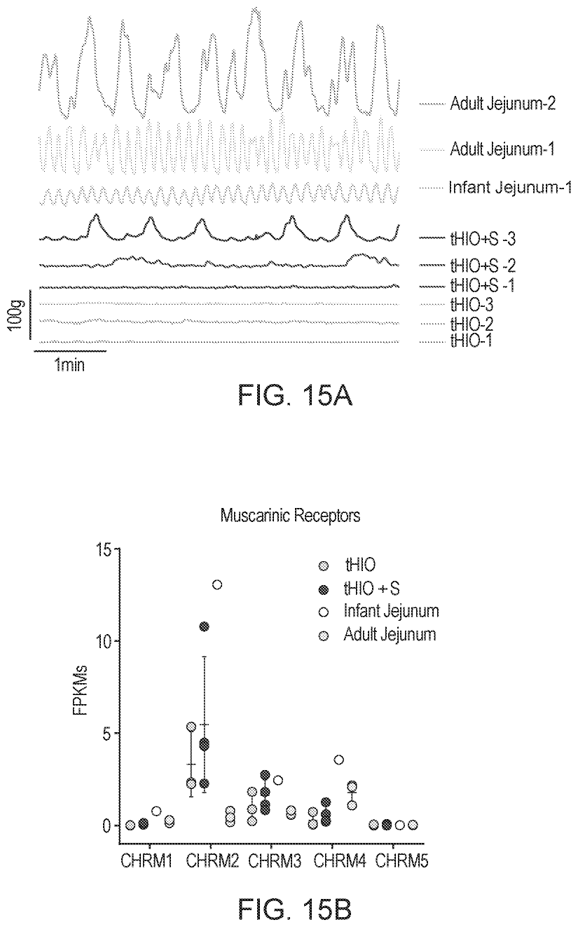

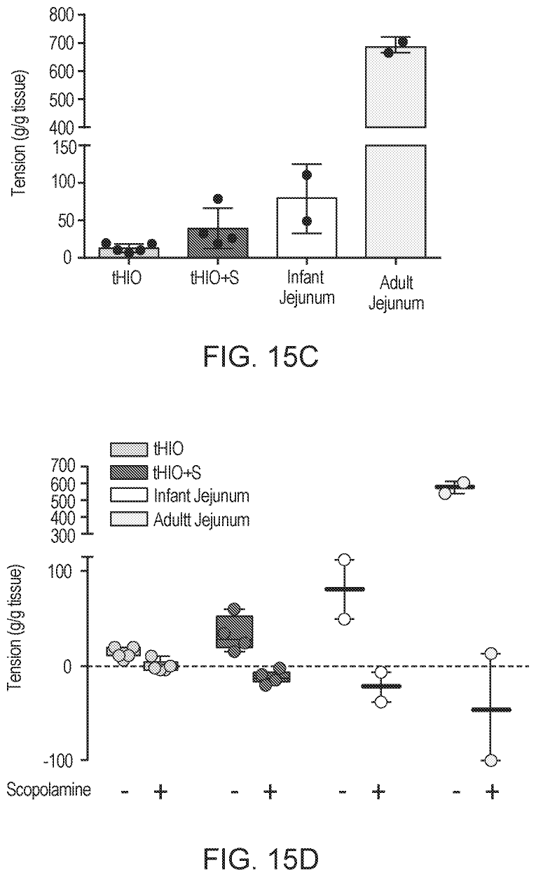

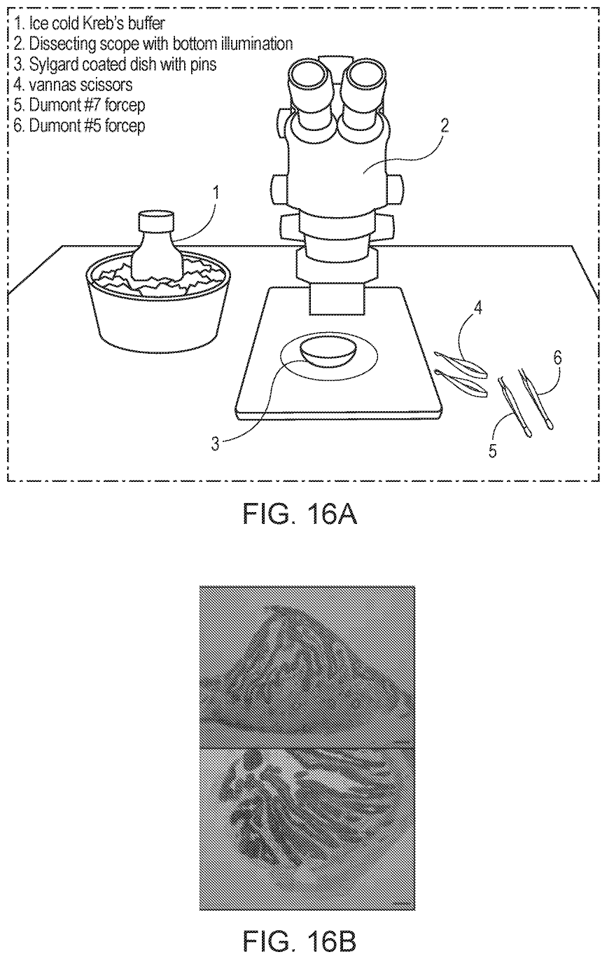

Applicant next tested ex vivo muscle properties to see if the increased muscle thickness further correlated to improved muscle function. In a previous study, the presence of intestinal cells of Cajal (ICCs) was confirmed in tHIOs⁵. Here, with Anoctamin1 (ANO1), a marker more specific for ICCs than CD117 which also stains mast cells, Applicant observed their presence in all sample types²⁴. In both tHIO and tHIO+S, ICCs were localized primarily within the smooth muscle layers, though not always at the interface of the longitudinal and circular muscle layers as was typically observed in adult jejunum (FIG. 7A). Spontaneous isometric contractions related to ICC activity in tHIO and tHIO+S were recorded and generally found to have a higher amplitude and duration in tHIO+S compared to tHIO (FIG. 7A, lower right). When observing isometric contractions in human jejunum this same association holds true, however due to the presence of the enteric nervous system they are not solely relatable to ICC activity. Thus, it was not surprising that they were observed to have a higher amplitude and frequency in comparison to PSC generated samples (FIG. 7A, lower right panel). A larger sampling of isometric force contractions were plotted to describe variability between biological repeats (FIG. 15A).

After an equilibration period, muscle force contraction was assayed using a logarithmic series of bethanechol doses ranging in concentration from 1 nM to 10 mM. Representative tensile response recordings are plotted for tHIO, tHIO+S and adult jejunum (FIG. 7B). From these data the effective concentration to achieve half of the maximal response (EC50) per tissue type was calculated (FIG. 7C, left panel). To better visualize the differences between tHIO and tHIO+S the data was also plotted sans adult jejunum (FIG. 7C, right panel). Due to such a low overall tensile response of tHIO to bethanechol, an EC50 with 95% confidence could not be determined. In tHIO+S, the response was more robust and an EC50 of 14.67 μM was calculated, which was much higher than that of adult human jejunum at 3.28 μM. Muscarinic receptor expression was confirmed transcriptionally in tHIO and tHIO+S, indicating that the decrease in reactivity was functional and not a reflection of poor receptor expression in PSC derived tissues (FIG. 11B). In calculating the maximal tension produced in tHIO and tHIO+S, Applicant observed the tHIO+S to have a significantly higher tensile capability (FIG. 7D). With the application of scopolamine, a nonspecific antimuscarinic, relaxation is observed in both tissues (FIG. 7E). These values were also measured in adult jejunum, which had a much greater tensile capacity than the PSC derived samples (FIG. 15C, 15D). Interestingly, when assaying infant jejunum, the responses were within the same order of magnitude as PSC derived samples (FIG. 15C, 15D). This suggests that significant muscle development occurs postnatally and supports that the maturation status of the tHIO+S is most similar to infant jejunum.

Discussion

Applicant has combined the principles of embryonic intestinal development with the mechanics of development to successfully engineer human pluripotent stem cell-derived intestinal tissue with maturity and function, which exceeds that of those produced with a strictly biological approach. Applicant has shown the application of strain induced gross, microscopic, and ultrastructural changes in the morphology of transplanted organoids making them more similar to native human samples. These structural changes were reflected transcriptionally, where a closer correlation to human tissue was observed with the application of strain. When testing function, Applicant also saw a positive effect of strain in the tHIO. Increasing trends in TER coupled with increases in tight junction protein transcripts suggest that barrier function increased as a result of strain. Applicant also observed an increase in overall muscle activity and tone indicating that strain promoted not only muscle growth, but strength as well. This dual approach to HIO generation seemed an improvement over the singular approach that is common in the field.

While the present study has not clearly elucidated the specific mechanism of mechanotransduction in the tHIO+S system that results in maturation, there is evidence that the TGFβ and MAPK pathways contribute as they were both enriched for transcriptionally. Protein analysis revealed an increase in TGFβ signaling activity as marked by an overall elevation in protein phosphorylation across the board, while the data was less convincing for MAPK signaling. However, with further investigation of pJUN, which is a transcription factor downstream in both MAPK and TGFβ signaling, expression was observed to be visually increased with strain by immunohistochemistry. Furthermore, pJUN has been described as one of the transcription factors controlling responses to mechanical strain in mechanotransduction pathways²⁵, making these pathways worthy candidates for investigation.

Vertebrate gut development and organogenesis involves not only specification and growth, which have been well described, but also mechanical processes²⁶⁻³⁰. Briefly, the gastrointestinal tract and its adjacent organs initially develop from an endodermal sheet, which folds to form a midline tube along the anterior-posterior axis of the embryo which can be divided into the foregut, midgut and hindgut regions²⁸. Following tube formation in humans is a significant elongation process which results in a hairpin fold, subsequent rotations and looping of the gut in the human embryo³¹. Evidence in animal models suggests that strain between the gut tube and mesentery, caused by differential growth rates, influences loop formation¹³. When this interaction is disrupted by detaching a portion of the mesentery from the gut tube looping fails to occur in that area of the gut¹³. Furthermore, the development and growth of the gut's smooth muscle layers have been shown to influence the epithelial architecture within the gut tube. Prior to the presence of smooth muscle, the luminal surface of the gut is smooth. It is with the development of the underlying circular muscle layer that longitudinal ridges and buckling of the epithelium occurs^{9,11}. These ridges go on to form zigzags, in chick, which ultimately resolve into villi with further growth of the longitudinal muscle layer^{9,11}. The growth of these muscle layers provides a compressive force limiting any outward development driving buckling of the luminal surface¹¹. These changes in geometry have been successfully modeled mathematically and found to hold true across several vertebrate species with slight variations in progression¹¹.

Not only does the gut experience an array of mechanical cues and forces during embryonic and fetal development, but is also undergoing mechanical manipulations postnatally. On a daily basis during the digestive process, the gut is experiencing internal and external forces. Both segmentation and peristalsis result in physical forces. Segmentation, attributed to focused coordinated contractions of the surrounding circular muscle, results in compressive force³². Peristaltic contractions proximal to the food bolus coupled with distal relaxation are then responsible for its forward movement, of which generates an internal sheer force and radial pressure on the epithelium^{32, 33}. Furthermore, it has been shown that an absence of mechano-luminal forces impact the gut homeostasis in humans³⁴. It is logical to believe the mechanical forces of digestion generated by the smooth muscle in coordination with the enteric nervous system play a role in the homeostasis of gut architecture, though this interplay has not been well described³⁴⁻³⁶.

With the advent of microfluidic devices, like the various “on-a-chip” bioreactors, Applicant is able to both model and modulate specific mechanical forces present in native tissues^{37,38}. This new category of devices may serve as a basis for a more developmental approach in constructing what Applicant might term as organogenesis chips, as they grow to include the biological, mechanical, and electrical facets required. Perhaps the well-described patterning protocols for deriving various organoids can be translated within a context of dynamic forces^{3,39-41}. Applicant would speculate that as the understanding of mimicry of natural developmental processes improves, the tissues yielded will better approximate native ones.

These experiments support the importance of creating interdisciplinary marriages between the biological and mechanical developmental processes in tissue engineering. The present study provides a platform which may be translated to bigger animal models to engineer more mature, larger scale human intestinal tissue segments, with the hopeful application of transplantation.

Methods

Human Tissue

Human tissue collection was performed with the prior approval of Cincinnati Children's Hospital Medical Center's (CCHMC) Institutional Review Board (Tissue Characterization, Study No. 2014-0427). Surgical samples of pathologically normal adult human jejunum were obtained from patients undergoing bariatric procedures between the ages of 16 and 25 years old. Informed consent or assent was obtained from all patients and/or parent/legal guardians as appropriate. Pathologically normal infant jejunum surgical samples and/or paraffin sections of ages three months and younger were obtained as de-identified samples from the Cincinnati Biobank, part of Cincinnati Children's Research Foundation as well as the Better Outcomes for Children Biorepository.

Animals

Adult immune-deficient NOD-SCID IL-2Rγnull (NSG) mice with ages between day of life 56 and 84 were used in all experiments (Comprehensive Mouse and Cancer Core Facility, Cincinnati, Ohio). Mice were housed in CCHMC's pathogen-free animal vivarium and handled humanely in accordance with the NIH Guide for the Care and Use of Laboratory Animals. NSG mice were fed antibiotic chow (275 p.p.m. Sulfamethoxazole and 1,365 p.p.m. Trimethoprim; Test Diet). Both food and water were provided ad libitum before and after surgeries. All experiments were performed with the prior approval of CCHMC's Institutional Animal Care and Use Committee (Signaling Pathways associated with Intestinal Stem Cell Expansion, Protocol No. 2016-0014).

Generation of Human Intestinal Organoids

Human Intestinal Organoids (HIOs) were generated and maintained as previously described²⁻⁴. Briefly, line H1 embryonic stem cells (WiCell Research Institute, Inc.) were grown in feeder-free conditions in Matrigel (BD Biosciences) coated six-well Nunclon surface plates (Nunc) and maintained in mTESR1 media (Stem Cell Technologies). For induction of definitive endoderm (DE), cells were passaged with Accutase (Stem Cell Technologies) and plated at a density of 65,000 cells per well in 24-well Nunc plates. Cells were allowed to grow in mTESR1 media for two days before treatment with 100 ng/ml of Activin A for three days as previously described. DE was then treated with hindgut induction medium (RPMI 1640, 100×NEAA, 2% dFCS), for four days with 100 ng/ml FGF4 (R&D) and 3 μM Chiron 99021 (Tocris) to induce formation of mid-hindgut spheroids. Spheroids were then plated in Growth Factor Reduced (GFR) Matrigel and maintained in intestinal growth medium (Advanced DMEM/F-12, N2 supplement, B27 supplement, 15 mM HEPES, 2 mM L-glutamine, penicillin-streptomycin) supplemented with 100 ng/ml EGF (R&D) to generate human intestinal organoids (HIOs). Media was changed twice weekly thereafter. HIOs were replated in fresh Matrigel every 14 days. HIOs were utilized for surgical transplantation between days 28 and 36.

Generation of Nitinol Springs

Nickel titanium (nitinol, NiTi) springs were formed as previously described²⁰. Briefly, NiTi wires of diameter 0.152 mm were wrapped around a mandrel and heat set in order to impart the spring's desired geometry (Nitinol Devices & Components). The resulting springs were then cut down to have a relaxed length of approximately 12 mm. Springs were compressed to half their relaxed length prior to implantation and placed within a gelatin capsule (Torpac, Inc.) which was subsequently double coated with C-A-P (Eastman Chemical Company) as previously described¹⁴.

Transplantation of Human Intestinal Organoids and Implantation of Springs

HIOs were prepared for transplantation as previously described^{4, 5, 42}. Briefly, single matrigel embedded HIOs were transplanted into the mesentery of the mice at the most distal arcade before the ileocecal junction. Mice were anesthetized with 2% inhaled isoflurane (Butler Schein), and the abdomen shaved and prepped in sterile fashion using isopropyl alcohol and povidine-iodine. A 2 cm midline incision was made and approximately 4 cm of the intestines pulled out. A small pocket was created in the mesentery and the HIO placed within. The abdominal cavity was irrigated with normal saline with Zosyn (2 mg/ml; Pfizer Inc.) and the intestine placed back within the abdominal cavity. The abdominal wall muscles and skin were then closed in a double layer fashion and the mice were given a subcutaneous injection of Buprenex (0.05 mg/kg; Midwest Veterinary Supply) for pain management. Ten to twelve weeks following engraftment, the mice then underwent a secondary surgery with similar preparations. During this procedure, the tHIO was incised along a length of approximately 3 mm using Vannas scissors to gain access to the lumen. Directionality of the incision varied between tHIOs based on their shape, vascularization and proximity to the mouse's bowel. Any mucous plugs were manually removed. Then, using an 18 G blunt tip fill needle positioned at the incision of the lumen, the tHIO was irrigated with normal saline. Care was taken during this process to avoid damaging the epithelium. With the luminal space cleared, the gelatin capsule was placed within the lumen of the tHIO to allow for spring deployment parallel to the adjacent mouse intestine. This was done to reduce the risk of obstruction. The tHIO was then closed using a 9-0 silk suture in a simple interrupted fashion. Sham operated mice underwent the same aforementioned procedure except that the C-A-P coated, gelatin capsule was empty, without spring. Mice were sacrificed and tissue harvested 14 d postoperatively.

MicroCT Imaging System

An IVIS Spectrum (PerkinElmer) coupled to a computer with Living Image software (Caliper Life Sciences) was used in the acquisition of microCT images. Briefly, live mice were anesthetized with 2% inhaled isoflurane prior to and during imaging which was used to monitor post-operative spring deployment.

Tissue Processing, Immunohistochemistry, and Light Microscopy

Segments of human jejunum, tHIO and tHIO+S grafts were fixed overnight in 4% paraformaldehyde (PFA), processed and embedded in paraffin. Sections of 5 μm thickness were deparaffinized and either stained immediately with a histological kit (Pentachrome, Newcomer Supply) or subjected to antigen retrieval, and stained. For both primary and secondary antibodies, incubations took place at 4° C. overnight in 1% bovine serum albumin in phosphate buffered saline (PBS). Antibodies, their respective dilutions are listed in Table 1. The Vectastain ABC system (Vector Laboratories) was used for amplification. The following kits were used for signal detection: diaminobenzidine substrate kit, Immpact SG substrate kit, and Vector Red substrate kit (Vector Laboratories). Lillie-Mayer's Hematoxylin (Dako North America, Inc.) or Nuclear Fast Red (PolySciences, Inc.) was used as a counterstain. Images were acquired using a Nikon Eclipse Ti microscope and analyzed using Nikon Elements Imaging Software (Nikon).

TABLE 1

| | | | | Company: |
|---|---|---|---|---|
| | Antigen | Dilution | Host | Catalog Number |
| Primary | ANO1 | 1:250 | rabbit | Abcam: ab53212 |
| | CDH1 | 1:500 | mouse | Abnova: PAB12286 |
| | CHGA | 1:500 | mouse | Abnova: H00009545- |
| | | | | A01 |
| | HuDPP4 | 1:1000 | goat | R&D: AF1180 |
| | HuKU80 | 1:200 | rabbit | Cell Signaling: 2180 |
| | MKI67 | 1:300 | rabbit | Abnova: PAB20694 |
| | HuLYZ | 1:750 | mouse | Atlas: AMAb90621 |
| | HuOLFM4 | 1:500 | rabbit | Cell Signaling: 14369 |
| | pJUN | 1:600 | rabbit | Cell Signaling: 3270 |
| | POU2F3 | 1:200 | rabbit | Atlas: HPA019652 |
| | SI | 1:1000 | rabbit | Thermo: HPA011897 |
| Secondary | α-goat biotin | 1:1000 | horse | Vector: BA-9500 |
| | α-mouse biotin | 1:1000 | horse | Vector: BA-2000 |
| | α-rabbit biotin | 1:1000 | goat | Vector: BA-1000 |

Electron Microscopy

Scanning Electron Microscopy. Segments of human jejunum, tHIO and tHIO+S grafts were fixed overnight in 3% gluteraldehyde in 0.175 M sodium cacodylate buffer with pH 7.4. Samples were then buffer rinsed and post fixed in 1% osmium tetroxide in 0.175 M cacodylate buffer for 1 hour at 4° C. After another buffer rinse, samples were put through a graded ethanol series (25, 50, 75, 95, 3×100%) for dehydration. Specimens were then critical point dried in a Leica EM CPD300, stub-mounted and sputter-coated 10 nm thick with 60/40 gold palladium using a Leica EM ACE600. A Hitachi SU8010 transmission electron microscope was used to image samples.

Transmission Electron Microscopy. Segments of human jejunum, tHIO and tHIO+S grafts were fixed and dehydrated in the same fashion as those for scanning electron microscopy. Infiltration was performed with 2× propylene oxide followed by a graded infiltration with LX-112. Samples were polymerized into blocks overnight at 37° C. and subsequently kept at 60° C. for 3 days. Thicks were sectioned and stained with Toluidine Blue to verify tissue orientation. Blocks were then sectioned at 10 nm, skipping 10 μm between grids. Sections were stained with urinyl acetate and lead citrate before imaging with a Hitachi H7600 transmission electron microscope.

Morphometric Analysis, Cell Type Quantification and Statistical Analysis

Morphometric analysis was performed on hematoxylin and eosin stained tissue sections. Crypt depth, crypt width, villus height, villus width, and mucosal thickness were measured for a minimum of 20 well-oriented crypt-villus units per tissue sample and then averaged using Nikon NIS imaging software (Nikon). Microvilli were measured using ImageJ; at least 150 microvilli per sample across three different grids of at least 10 μm apart were quantified. Profiles of proliferation by position within the crypt were determined using dual MKI67 and CDH1 immunostaining. A minimum of 10 intact crypts were analyzed per sample and averaged. For quantification of cell types along the crypt-villus axis the following were utilized: CHGA immunostaining for quantification of endoendocrine cells, Alcian Blue pH 2.5 staining for goblet cells, LYZ for Paneth cells. For semi-quantitative analysis of the stem cell compartment OLFM4 immunostaining was used. In each case a minimum of 20 well-oriented crypt-villus units per tissue sample were quantified and averaged. All data are represented as mean±standard deviation. For analysis of proliferative profiles, Gaussian curves were fit to each data set and a comparison of fits was performed using Prism software (GraphPad). Two-way ANOVAs and post-hoc Tukey's tests were completed using Prism software as appropriate. The statistical significance cutoff was p<0.05 and is further denoted as follows: *=0.01<p<0.05, **=0.001<p<0.01, ***=p<0.001, and ****=p<0.0001.

Ex Vivo Epithelial Permeability

The epitheliums of freshly harvested tHIO, tHIO+S grafts and human surgical samples were carefully dissected through a technique similar to seromuscular stripping23, 43. tHIOs and fresh surgical samples were opened longitudinally. All further dissection was done in ice cold Kreb's buffer (NaCl, 117 mM; KCl, 4.7 mM; MgCl₂, 1.2 mM; NaH₂PO₄, 1.2 mM; NaHCO₃, 25 mM; CaCl₂, 2.5 mM and glucose, 11 mM). Tissue segments were then pinned in a dish containing 0.5 cm thick cured Sylgard (Electron Microscopy Sciences). Unlike mouse tissue, the seromusculature layers (serosa, longitudinal, and circular smooth muscle) are relatively thick in both tHIOs and human jejunum. These layers are then micro-dissected as one unit from the epithelium using Dumont #5 and #7 forceps along with Vannas scissors taking care to only handle the edges of the tissue (Fine Science Tools, Inc.). The layers are gently separated and cut in small increments. After separation, gross tissue integrity was assessed using the stereoscope's bottom lighting for evenness and uniformity in appearance. The edges of the tissue, experiencing the majority of manipulation, were discarded along with any portions of the epithelium that appeared damaged. The dissection set up can be found in FIG. 6A. Two examples of tissues dissected in this fashion can be found in FIG. 6B after completion of the Ussing assay. Some remnant subepithelial mucosa remained after dissection. Epithelial segments were mounted between the hemi-chambers of an Ussing apparatus (Physiologic Instruments), and 0.008 cm2 of tissue was exposed to 3 mL of oxygenated Krebs buffer at 37° C. The transepithelial potential difference was detected with two paired electrodes containing 4% agar in 3 M KCl. The electrodes were connected to a VVC MC8 voltage clamp amplifier (Physiologic Instruments, San Diego). Electrode potential difference and fluid resistance values were offset before tissue segments were mounted in the chamber. A 30 min monitoring period was allowed for the establishment of equilibrium in the chamber. Then, tissues were voltage-clamped at 0 mV while continuously measuring the short circuit current (Isc). For FITC-dextran permeability, 2.2 mg/ml FITC-dextran was added into apical side, and a sample was taken from the basolateral side every 30 minutes for 3 hours, replacing the same amount of fresh modified Kreb's buffer in the basolateral side. Finally, samples were read with a plate-reader (Synergy 2, BioTek).

Ex Vivo Muscle Strength

tHIO and tHIO+S grafts were harvested and placed in ice-cold Hank's Balanced Salt Solution (HBSS). Human surgical samples were maintained overnight at 4° C. in HBSS prior to assaying in order to minimize the effects of anesthetics. Muscle strips (4-6 mm in length and 1-2 mm in width) were dissected from the samples. Strips were then suspended vertically in an organ bath chamber (Radnoti) filled with freshly prepared Krebs-Ringer solution (Sigma; supplemented with 2.5 mM CaCl and 15 mM NaHCO₃; pH 7.4), warmed to 37° C. and gassed with 95% O₂+5% CO₂. Segments were allowed to equilibrate for an hour at an initial tension of 0.5 g. The contractile response of the muscle was continuously recorded, using 4-chamber tissue-organ bath chambers with isometric force transducers (0-25 g; AD Instruments) coupled to a multi-channel bridge amplifiers and data recorder (AD Instruments; PowerLab 4/35) linked to a computer equipped with LabChart Pro software (AD Instruments). A logarithmic dose response to Carbamyl-p-methylcholine chloride (Bethanechol; Sigma-Aldrich) was obtained through the administration of exponential doses with concentrations of 1 nM to 10 mM at 2 minute intervals before the administration of 10 μM scopolamine (Tocris Bioscience).

RNA Isolation and Sequencing

RNA was extracted using an RNeasy Plus Micro Kit (Qiagen) according to manufacturer guidelines. Samples were then quantified and submitted to CCHMC's DNA Sequencing and Genotyping core for Next Generation Sequencing. All samples were assayed to have RNA integrity numbers greater than eight. After quality control, a cDNA library was created and sequenced using an IlluminaHiSeq2000 (Illumina) with 20 million paired-end reads per sample.

RNA Sequencing Bioinformatics

Pre-Processing

Data pre-processing was performed in Galaxy public server (https://usegalaxy.org). fastq datasets were encoded in Phred+33 to fastqsanger under the attributes for each dataset (FASTQ Groomer, Galaxy v1.0.4). Read quality was assessed using FastQC (Galaxy, v0.67). Adapter and low quality (<20) sequences were removed using Trimmomatic (SLIDINGWINDOW) (Galaxy v0.32.3). Reads were mapped against the human genome GRCh37Hg19 using Tophat2/Bowtie2 with default settings (Galaxy v2.1.0). The number of reads uniquely mapping to each gene was counted using feature Counts (Galaxy v1.4.6.p5) using the Illumina iGenomes (http://support.illumina.com/sequencing/sequencing_software/igenome.ilmn) annotation file. Alternatively, each sample was independently processed with Cufflinks⁴⁴ in order to generate an initial transcriptome. Applicant used the Cuffmerge tool to merge the private transcriptomes into a single reference, and at the same time annotated known genes and extended partial transcripts⁴⁵. This common transcriptome was used in a second pass with Cufflinks to quantify each transcript and gene (known or novel) in each sample⁴⁶. The reference annotation used was based on the UCSC knownGenes table⁴⁷.

Analysis of RNA-Seq Data

RNA-seq analysis was performed using Strand NGS 2.9 software (Strand Life Sciences). Count tables generated in Galaxy were processed and normalized using the DESeq2 package⁴⁸ within the Strand NGS 2.9 R console. Applicant performed a PCA using Strand NGS 2.9 software. From the PCA, the gene with the highest loadings for each principal component were extracted and plotted using the standalone hi_loadings function in pcaExplorer package. A functional enrichment analysis using the limmaquickpca2go routine provided by the limma package was performed on the 10000 genes with the highest loadings for each principal component. ANOVA and moderated t-Test were used to perform statistical analysis amongst samples and between groups. Functional enrichment analysis was performed in the ToppGene suite (https://toppgene.cchmc.org). Plots were generated using ggplot2 v2.2.1 and GOplot v1.02 in Rstudio v1.0.14.

Statistics on FPKM Data

For statistics on normalized FPKMs, either a Student T test or Mann-Whitney test was used according to data distribution. The statistical significance cutoff was p<0.05 and is further denoted as follows: *=0.01<p<0.05, **=0.001<p<0.01, ***=p<0.001, and ****=p<0.0001.

Analysis of RNA-Seq Data with Publicly Available Datasets

Publicly available datasets of fetal and adult full thickness intestinal tissues were downloaded from EBI-AE and NCBI-GEO databases using the Galaxy public server (Table 2). Datasets were selected upon RNA extraction and library preparation methods and sequencing platform (Illumina HiSeq). Only forward reads were used from paired-end files. Datasets were encoded to fastqsanger and read quality was assessed using FastQC (Galaxy, v0.67). Adapter and low quality (<20) sequences were removed using Trimmomatic (SLIDINGWINDOW). Reads were mapped against the human genome GRChb37\Hgl9 using HISAT2 with default settings (Galaxy v2.0.5.1)⁴⁹. Count tables were generated in Galaxy using feature Counts using the Illumina iGenomes annotation file. The count matrix was processed in R 3.4.1 using the functions exported by the pcaExplorer package50 for a standalone usage. Briefly, the count matrix was transformed and normalized using a variance stabilizing transformation (VST) to the count data. Applicant visualized the sample PCA using the pcaplot function. The genes with the highest variance were selected to compute the PCA i.e. from 500 to 10000 genes. From the PCA, the gene with the highest loadings for each principal component were extracted and plotted using the hi_loadings function. A functional enrichment analysis using the limmaquickpca2go routine provided by the limma package was performed on the 10000 genes with the highest loadings for each principal component. The detailed analysis and R scripts are provided in the following Github repository (www.github.com\maximemahe\Poling2017). The accession number for data generated for this paper is ArrayExpress: E-MTAB-6017 and includes the transplanted HIO, with or without lengthening device, the human adult and infant data shown in FIG. 3 and FIG. 9. The additional RNA-seq datasets including HIOs derived from H1 and H9 ES cell lines, transplanted HIOs, Fetal, newborn and adult small intestine were downloaded from public databases (Table 3)

TABLE 2
Antibodies and dilutions for immunohistochemistry.

| | | | | Company: |
|---|---|---|---|---|
| | Antigen | Dilution | Host | Catalog Number |
| Primary | ANO1 | 1:250 | rabbit | Abcam: ab53212 |
| | CDH1 | 1:500 | mouse | Abnova: PAB12286 |
| | CHGA | 1:500 | mouse | Abnova: H00009545- |
| | | | | A01 |
| | HuDPP4 | 1:1000 | goat | R&D: AF1180 |
| | HuKU80 | 1:200 | rabbit | Cell Signaling: 2180 |
| | MKI67 | 1:300 | rabbit | Abnova: PAB20694 |
| | HuLYZ | 1:750 | mouse | Atlas: AMAb90621 |
| | HuOLFM4 | 1:500 | rabbit | Cell Signaling: 14369 |
| | pJUN | 1:600 | rabbit | Cell Signaling: 3270 |
| | POU2F3 | 1:200 | rabbit | Atlas: HPA019652 |
| | SI | 1:1000 | rabbit | Thermo: HPA011897 |
| Secondary | α-goat biotin | 1:1000 | horse | Vector: BA-9500 |
| | α-mouse biotin | 1:1000 | horse | Vector: BA-2000 |
| | α-rabbit biotin | 1:1000 | goat | Vector: BA-1000 |

TABLE 3
RNAseq Datasets Downloaded from Public Databases.

| Sample Label | Sample Description | Tissue |
|---|---|---|
| Fetal_SI_2 | Fetal Intestine, Small; RNA.RS18147 | Fetal tissue |
| Fetal_SI_3 | Fetal Intestine, Small; RNA.RS18148 | Fetal tissue |
| Fetal_SI_4 | Fetal Intestine, Small; RNA.RS18763 | Fetal tissue |
| Fetal_SI_5 | Fetal Intestine, Small; RNA.RS18765 | Fetal tissue |
| Fetal_SI_6 | Fetal Intestine, Small; RNA.RS18767 | Fetal tissue |
| Infant_SI_1 | Control patient 1 | Infant |
| Infant_SI_2 | Control patient 2 | Infant |
| Infant_SI_3 | Control patient 3 | Infant |
| Infant_SI_4 | Control patient 4 | Infant |
| Infant_SI_5 | Control patient 5 | Infant |
| Child_SI_1 | Small Bowel Cells; polyA-RNA-seq_STL001SB_r1a | Child |
| Adult_SI_2 | Small Bowel Cells; polyA-RNA-seq_STL002SB_r1a | Adult |
| Adult_SI_3 | smallintestine_4a | Adult |
| Adult_SI_4 | smallintestine_4b | Adult |
| Adult_SI_5 | smallintestine_4c | Adult |
| Adult_DUO_1 | duodenum_4b | Adult |
| Adult_DUO_2 | duodenum_4c | Adult |

| Sample Label | Tissue Region | Age | Gender | Repository | Database # | Accession # |
|---|---|---|---|---|---|---|
| Fetal_SI_2 | Small Intestine | GW 15.4 | Female | NCBI-GE O | GSE 18927 | GSM1059507 |
| Fetal_SI_3 | Small Intestine | GW 13 | Male | NCBI-GE O | GSE 18927 | GSM1059508 |
| Fetal_SI_4 | Small Intestine | GW 16.4 | Male | NCBI-GE O | GSE 18927 | GSM1059517 |
| Fetal_SI_5 | Small Intestine | GW 17.1 | Female | NCBI-GE O | GSE 18927 | GSM1059519 |
| Fetal_SI_6 | Small Intestine | GW 14 | Female | NCBI-GE O | GSE 18927 | GSM1059521 |
| Infant_SI_1 | Ileum | GW 33 | Female | NCBI-GE O | GSE 64801 | GSM1580784 |
| Infant_SI_2 | Ileum | GW 26 | Female | NCBI-GE O | GSE 64801 | GSM1580785 |
| Infant_SI_3 | Ileum | GW 26 | Male | NCBI-GE O | GSE 64801 | GSM1580786 |
| Infant_SI_4 | Ileum | GW 33 | Male | NCBI-GE O | GSE 64801 | GSM1580787 |
| Infant_SI_5 | Ileum | GW 39 | Female | NCBI-GE O | GSE 64801 | GSM1580788 |
| Child_SI_1 | Small Intestine | 3 yrs | Male | NCBI-GE O | GSE 16256 | GSM1010940 |
| Adult_SI_2 | Small Intestine | 30 yrs | Female | NCBI-GE O | GSE 16256 | GSM1120313 |
| Adult_SI_3 | Small Intestine | N/A | N/A | E BI-AE | E-MTAB-1733 | ERS326961 |
| Adult_SI_4 | Small Intestine | N/A | N/A | E BI-AE | E-MTAB-1733 | ERS326971 |
| Adult_SI_5 | Small Intestine | N/A | N/A | E BI-AE | E-MTAB-1733 | ERS326958 |
| Adult_DUO_1 | Duodenum | N/A | N/A | E BI-AE | E-MTAB-1733 | ERS326992 |
| Adult_DUO_2 | Duodenum | N/A | N/A | E BI-AE | E-MTAB-1733 | ERS326976 |

Source for all Tissue samples = fresh.

Protein Phosphorylation Arrays

C-Series phosphorylation arrays were performed according to manufacturer recommendations (RayBiotech, Inc). Briefly, protein was extracted from flash frozen full thickness tHIO and tHIO+S tissues, quantified, and normalized between samples. Protein was incubated on antibody array nitrocellulose membranes, followed by horseradish peroxidase based amplification and detection. Chemiluminescent readings were taken using a ChemiDoc MP imaging system (Bio-Rad Laboratories, Inc.) and densitometry data extracted using ImageJ software. Readings were normalized to the positive loading controls and membrane background signal subtracted.

### REFERENCES

1. Wells, J. M. & Spence, J. R. How to make an intestine. Development 141, 752-760 (2014).
2. Spence, J. R. et al. Directed differentiation of human pluripotent stem cells into intestinal tissue in vitro. Nature 470, 105-109 (2011).
3. McCracken, K. W., Howell, J. C., Wells, J. M. & Spence, J. R. Generating human intestinal tissue from pluripotent stem cells in vitro. Nature protocols 6, 1920-1928 (2011).
4. Watson, C. L. et al. An in vivo model of human small intestine using pluripotent stem cells. Nature medicine 20, 1310-1314 (2014).
5. Workman, M. J. et al. Engineered human pluripotent-stem-cell-derived intestinal tissues with a functional enteric nervous system. Nature medicine 23, 49-59 (2017).
6. Finkbeiner, S. R. et al. Transcriptome-wide Analysis Reveals Hallmarks of Human Intestine Development and Maturation In Vitro and In Vivo. Stem cell reports (2015).
7. Dedhia, P. H., Bertaux-Skeirik, N., Zavros, Y. & Spence, J. R. Organoid Models of Human Gastrointestinal Development and Disease. Gastroenterology 150,1098-1112 (2016).
8. Yu, H. et al. The Contributions of Human Mini-Intestines to the Study of Intestinal Physiology and Pathophysiology. Annu Rev Physiol 79, 291-312 (2017).
9. Nelson, C. M. On Buckling Morphogenesis. J Biomech Eng 138, 021005 (2016).
10. Kurpios, N. A. et al. The direction of gut looping is established by changes in the extracellular matrix and in cell:cell adhesion. Proceedings of the National Academy of Sciences of the United States of America 105, 8499-8506 (2008).
11. Shyer, A. E. et al. Villification: how the gut gets its villi. Science 342, 212-218 (2013).
12. Shyer, A. E., Huycke, T. R., Lee, C., Mahadevan, L. & Tabin, C. J. Bending gradients: how the intestinal stem cell gets its home. Cell 161, 569-580 (2015).
13. Savin, T. et al. On the growth and form of the gut. Nature 476, 57-62 (2011).
14. Stark, R., Panduranga, M., Carman, G. & Dunn, J. C. Development of an endoluminal intestinal lengthening capsule. Journal of pediatric surgery 47, 136-141 (2012).
15. Rouch, J. D. et al. Scalability of an endoluminal spring for distraction enterogenesis. Journal of pediatric surgery 51, 1988-1992 (2016).
16. Demehri, F. R. et al. Development of an endoluminal intestinal attachment for a clinically applicable distraction enterogenesis device. Journal of pediatric surgery 51, 101-106 (2016).
17. Demehri, F. R., Freeman, J. J., Fukatsu, Y., Luntz, J. & Teitelbaum, D. H. Development of an endoluminal intestinal lengthening device using a geometric intestinal attachment approach. Surgery 158, 802-811 (2015).
18. Luntz, J., Brei, D., Teitelbaum, D. & Spencer, A. Mechanical Extension Implants for Short-Bowel Syndrome. Proc SPIE Int Soc Opt Eng 6173, 617309 (2006).
19. Allard, J. et al. Immunohistochemical toolkit for tracking and quantifying xenotransplanted human stem cells. Regenerative medicine 9, 437-452 (2014).
20. Shekherdimian, S., Panduranga, M. K., Carman, G. P. & Dunn, J. C. The feasibility of using an endoluminal device for intestinal lengthening. Journal of pediatric surgery 45, 1575-1580 (2010).
21. Marsh, M. N. & Swift, J. A. A study of the small intestinal mucosa using the scanning electron microscope. Gut 10, 940-949 (1969).
22. Hooton, D., Lentle, R., Monro, J., Wickham, M. & Simpson, R. The Secretion and Action of Brush Border Enzymes in the Mammalian Small Intestine. Rev Physiol Biochem Pharmacol 168, 59-118 (2015).
23. Clarke, L. L. A guide to Ussing chamber studies of mouse intestine. American journal of physiology. Gastrointestinal and liver physiology 296, G1151-1166 (2009).
24. Gomez-Pinilla, P. J. et al. Ano1 is a selective marker of interstitial cells of Cajal in the human and mouse gastrointestinal tract. Am J Physiol Gastrointest Liver Physiol 296, G1370-1381 (2009).
25. Mammoto, A., Mammoto, T. & Ingber, D. E. Mechanosensitive mechanisms in transcriptional regulation. Journal of cell science 125, 3061-3073 (2012).
26. Burn, S. F. & Hill, R. E. Left-right asymmetry in gut development: what happens next? Bioessays 31, 1026-1037 (2009).
27. Sherwood, R. I., Chen, T. Y. & Melton, D. A. Transcriptional dynamics of endodermal organ formation. Developmental dynamics: an official publication of the American Association of Anatomists 238, 29-42 (2009).
28. Zorn, A. M. & Wells, J. M. Vertebrate endoderm development and organ formation. Annu Rev Cell Dev Biol 25, 221-251 (2009).
29. Spence, J. R., Lauf, R. & Shroyer, N. F. Vertebrate intestinal endoderm development. Developmental dynamics: an official publication of the American Association of Anatomists 240, 501-520 (2011).
30. Shahbazi, M. N. et al. Self-organization of the human embryo in the absence of maternal tissues. Nature cell biology 18, 700-708 (2016).
31. Soffers, J. H. M., Hikspoors, J. P. J. M., Mekonen, H. K., Koehler, S. E. & Lamers, W. H. The growth pattern of the human intestine and its mesentery. Bmc Dev Biol 15 (2015).
32. Tran, K., Brun, R. & Kuo, B. Evaluation of regional and whole gut motility using the wireless motility capsule: relevance in clinical practice. Therap Adv Gastroenterol 5, 249-260 (2012).
33. Engmann, J. & Burbidge, A. S. Fluid mechanics of eating, swallowing and digestion—overview and perspectives. Food & function 4, 443-447 (2013).
34. Wieck, M. M. et al. Prolonged Absence of Mechanoluminal Stimulation in Human Intestine Alters the Transcriptome and Intestinal Stem Cell Niche. Cell Mol Gastroenterol Hepatol 3, 367-388 e361 (2017).
35. Terry, B. S., Lyle, A. B., Schoen, J. A. & Rentschler, M. E. Preliminary mechanical characterization of the small bowel for in vivo robotic mobility. J Biomech Eng 133, 091010 (2011).
36. Gregersen, H., Kassab, G. S. & Fung, Y. C. The zero-stress state of the gastrointestinal tract: biomechanical and functional implications. Digestive diseases and sciences 45, 2271-2281 (2000).
37. Low, L. A. & Tagle, D. A. Organs-on-chips: Progress, challenges, and future directions. Exp Biol Med (Maywood), 1535370217700523 (2017).
38. Christoffersson, J., van Noort, D. V. & Mandenius, C. F. Developing organ-on-a-chip concepts using bio-mechatronic design methodology. Biofabrication (2017).
39. Sato, T. & Clevers, H. SnapShot: Growing Organoids from Stem Cells. Cell 161 (2015).
40. McCracken, K. W. et al. Modelling human development and disease in pluripotent stem-cell-derived gastric organoids. Nature 516, 400-+(2014).
41. Grapin-Botton, A. Three-dimensional pancreas organogenesis models. Diabetes Obes Metab 18, 33-40 (2016).
42. Mahe, M. M., Brown, N. E., Poling, H. M. & Helmrath, M. A. In vivo Model of Small Intestine. Methods Mol Biol 1597, 229-245 (2017).
43. Giles, D. A. et al. Thermoneutral housing exacerbates nonalcoholic fatty liver disease in mice and allows for sex-independent disease modeling. Nature medicine 23, 829-838 (2017).
44. Trapnell, C. et al. Transcript assembly and quantification by RNA-Seq reveals unannotated transcripts and isoform switching during cell differentiation. Nat Biotechnol 28, 511-515 (2010).
45. Roberts, A., Pimentel, H., Trapnell, C. & Pachter, L. Identification of novel transcripts in annotated genomes using RNA-Seq. Bioinformatics 27, 2325-2329 (2011).
46. Roberts, A., Trapnell, C., Donaghey, J., Rinn, J. L. & Pachter, L. Improving RNA-Seq expression estimates by correcting for fragment bias. Genome Biol 12, R22 (2011).
47. Hsu, F. et al. The UCSC Known Genes. Bioinformatics 22, 1036-1046 (2006).
48. Love, M. I., Huber, W. & Anders, S. Moderated estimation of fold change and dispersion for RNA-seq data with DESea2. Genome Biol 15, 550 (2014).
49. Kim, D., Langmead, B. & Salzberg, S. L. HISAT: a fast spliced aligner with low memory requirements. Nat Methods 12, 357-360 (2015).
50. Marini, F. pcaExplorer: Interactive Visualization of RNA-seq Data Using a Principal Components Approach. R package version 2.3.0. (2017).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of enhancing development of a three-dimensional in vitro model of an intestinal tissue (HIO) derived from a precursor cell comprising a lumen, comprising inserting a lengthening device into said lumen, wherein said lengthening device applies strain to said lumen.

2. The method of claim 1, wherein said lengthening device comprises a cavity for the flow of lumen secretions.

3. The method of claim 1, wherein said method comprises a first and a second engraftment period, wherein said first engraftment period comprises transplanting said HIO into an immune-compromised animal model prior to implantation of said lengthening device said HIO develops crypt regions, villi, and smooth muscle layers.

4. The method of claim 3, wherein said second engraftment period occurs after said first engraftment period, wherein said second engraftment period comprises inserting said lengthening device after said first engraftment period until villus height and crypt depth is increased as compared to a control HIO not subjected to said lengthening device.

5. The method of claim 3, wherein said first engraftment period is carried out until said HIO obtains a blood supply.

6. The method of claim 3, wherein said first and second engraftment period is carried out until said HIO has increased villus height and crypt depth and crypt fission, and increased longitudinal and circular muscle thickness, as compared to a control HIO which does not contain a lengthening device.

7. The method of claim 1, wherein said lengthening device has a relaxed length of about 10 to about 15 mm.

8. The method of claim 1, wherein said lengthening device has a compressed length of about 4 to about 8 mm.

9. The method of claim 1, wherein said lengthening device has a diameter of about 1 to about 3 mm.

10. The method of claim 1, wherein said lengthening device has a spring constant of from about 0.5 N/m to about 2 N/m.

11. The method of claim 1, wherein said precursor cell is a pluripotent stem cell or an induced pluripotent stem cell.

12. The method of claim 1, wherein said HIO is derived from a pluripotent stem cell, wherein said pluripotent stem cell is derived from a fetal tissue stem cell.

13. A three-dimensional human intestinal organoid (HIO) composition derived from a pre-cursor cell in vitro, wherein said HIO comprises a lengthening device.

14. A three-dimensional human intestinal organoid (HIO) composition derived from a pre-cursor cell in vitro, wherein said HIO is lacking one or more features native to a fully developed organ.

15. (canceled)
